SEQUENCE LISTING (89 sequences)

SEQ ID NO: 1 - protein, 116 aa, synthetic construct
QVQLVQSGGGVVQPGRSLRLACAASGLTVNNYAMHWVRQAPGKGLEWVAVISYDGNNKYYADSVKGRFTISRDNSKNTLYLQMNSLRAEDTAVYYCAKGGGYFDLWGRGTLVTVSS

SEQ ID NO: 2 - protein, 116 aa, synthetic construct
QVQLVQSGGGVVQPGRSLRLACAASGFTVNNYAMHWVRQAPGKGLEWVAVISYDGNNKYYADSVKGRFTISRDNSKNTLYLQMNSLRAEDTAVYYCAKGGGYFDLWGRGTLVTVSS

SEQ ID NO: 3 - protein, 116 aa, synthetic construct; identical to SEQ ID NO: 2

SEQ ID NO: 4 - protein, 116 aa, synthetic construct
QVQLQQSGGGVVQPGRSLRLSCAASGFTFSSYAMHWVRQAPGKGLEWVAFIRSDGSKKYYADSVKGRFTISRDNSKNTLYLQMNSLRAEDTAVYYCARHGNYFDSWGQGTLVTVSS

SEQ ID NO: 5 - protein, 120 aa, synthetic construct
QVQLVESGGGVVQPGRSLRLSCAASGFTFSTYGMHWVRQAPGKGLEWLSFISYDGDEKYYADSVKGRFTISRDNSKNTLYLQMNSLRAEDTAVYWCAKASGYGMGILDYWGQGTLVTVSS

SEQ ID NO: 6 - protein, 121 aa, synthetic construct
EVQLVESGGGLVQPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRRYWGQGTLVTVSS

SEQ ID NO: 7 - protein, 121 aa, synthetic construct
QVQLQESGGGVVRPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRHYWGQGTLVTVSS

SEQ ID NO: 8 - protein, 121 aa, synthetic construct
EVQLVESGGGLVQPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRHYWGQGTLVTVSS

SEQ ID NO: 9 - protein, 127 aa, synthetic construct
QVQLQESGGGLVQPGGSLRLSCAASGFTFSSYWMSWVRQAPGKGLEWVADIKQDGSEKYYVDSVKGRFTISGDNAKNSLYLQMNSLRAEDTAVYYCAKDVGSTAINYVRAYTWFDPWGQGTLVTVSS

SEQ ID NO: 10 - protein, 122 aa, synthetic construct
QVQLQESGGGLVQPGGSLRLSCAASGFTFSNYAMSWVRQAPGKGLEWVSTISGSGSSTFYVDSVKGRFTISRDNSKNTLYLQMNSLRAEDTAVYYCAQGLYSSGWANWFDPRGQGTLVTVSS

SEQ ID NO: 11 - protein, 123 aa, synthetic construct
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCAKVMGLAAAGLDAFDIWGQGTTVTVSS

SEQ ID NO: 12 - protein, 117 aa, synthetic construct
QVQLVQSGGGVVQPGRSLRLSCAASGFTFSSYAMHWVRQAPGKGLEWVAVISYDGSNKYYADSVKGRFTISRDTSTNTLYLQMNSLRADDTAVYYCGRESSGSPGVWGQGTTVTVSS

SEQ ID NO: 13 - protein, 122 aa, synthetic construct
QVQLVESGGGLIQPGGSLRLSCAASGFTVSSNYMSWVRQAPGKGLEWVSVIYTDGSTYYADSVKGRFTISRDNSKNTLYLQMNSLRAEDTAIYYCARDRGTSGYDWAWFDLWGQGTLVTVSS

SEQ ID NO: 14 - protein, 123 aa, synthetic construct
QVQLQESGGGLVQPGGSLRLSCAASGFTFSSYAMSWVRQAPGKGLEWVSAISGSGGSTYYADSVKGRFTISRDNSKNTLYMQMNSLRAEDTAVYYCAKDRYYYGSGKDAFDIWGRGTMVTVSS

SEQ ID NO: 15 - protein, 118 aa, synthetic construct
QVQLVESGGGLVQPGGSLGLSCAASGFTFSNYWMSWVRQAPGKGLEWVANVRQDGGQKYYVDSVKGRFTISRDNAKNSLYLQMNSLRTEDTAVYFCVSQRNSGEHDYWGQGTLVTVSS

SEQ ID NO: 16 - protein, 121 aa, synthetic construct; identical to SEQ ID NO: 8

SEQ ID NO: 17 - protein, 121 aa, synthetic construct
EVQLVESGGGLVQPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRNYWGQGTLVTVSS

SEQ ID NO: 18 - protein, 121 aa, synthetic construct; identical to SEQ ID NO: 7

SEQ ID NO: 19 - protein, 121 aa, synthetic construct
EVQLVESGGGLVKPGGSLRLSCAASGFTFSDYYMSWIRQAPGKGLEWVSYISSSGSSIYYADSVKGRFTISRDNAKNSLYLQMNSLKAEDTAVYYCARDITDVVGVSFDYWGQGTLVTVSS

SEQ ID NO: 20 - protein, 123 aa, synthetic construct
EVQLVESGGGVVQPGRSLRLSCAASGFTFSSYGMHWVRQAPGKGLEWVAVISYDGSNKYYADSVKGRFTISRDNSKNTLYLQMNSLRAEDTAEYYCAKVMGLAAAGLDAFDIWGQGTLVTVSS

SEQ ID NO: 21 - protein, 123 aa, synthetic construct
EVQLVESGGGVVQPGRSLRLSCAASGFTFSSFGMHWVRRAPGKGLEWVAVISYDGSNQYYADSVKGRFTISRDNSKNTLYLQMNSLRAEDTAVYYCGSRPGGGYASGSTVAYWGQGTLVTVSS

SEQ ID NO: 22 - protein, 109 aa, synthetic construct
QSVLTQPPSVSGAPGQRVTISCTGSSSNIGAGYDVHWYQQLPGTAPKLLIYGNNNRPSGVPDRFSGSKSGTSASLAITGLQAEDEADYYCSSYTSGTWLFGGGTKLTVL

SEQ ID NO: 23 - protein, 109 aa, synthetic construct; identical to SEQ ID NO: 22

SEQ ID NO: 24 - protein, 109 aa, synthetic construct
QSVLTQPPSVSGAPGQRVTISCTGSSSNIGAGYDVHWYQQLPGTAPKLLIYGDNNRPSGVPDRFSGSKSGTSASLAITGLQAEDEADYYCSSYTSGTWLFGGGTKLTVL

SEQ ID NO: 25 - protein, 107 aa, synthetic construct
DIQMTQSPSFLSASVGDRVTITCRASQGISSYLAWYQQKPGKAPKLLIYAASTLQSGVPSSFSGSGSGTEFTLTISSLQPEDFATYYCQQLASYPLTFGGGTKVDIK

SEQ ID NO: 26 - protein, 109 aa, synthetic construct
SSELTQDPAVSVALGQTVRITCQGDSLRSYYVSWFQQKPGQAPVFVMYGQNNRPSGISERFSGSSSGNTASLIITGAQAEDEADYYCHSRDSSGTHLRVFGGGTKLTVL

SEQ ID NO: 27 - protein, 110 aa, synthetic construct
QSALTQPPSASATPGQRVTISCSGRTSNIGSNHVYWYQQLPGTAPKLLIYRNNQRPSGVPDRFSGSKSGTSASLAISGLRSEDEADYYCATWDDSLSGEVFGGGTKLTVL

SEQ ID NO: 28 - protein, 109 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGTHLEVFGGGTKVTVL

SEQ ID NO: 29 - protein, 107 aa, synthetic construct
SSELTQDPAVSVALGQTVRITCQGDTLSTYYANWYQQKPGQAPVLVIYGKNNRPSGIPDRFSGSSSGNTASLTITGAQAEDEADYYCHSRDISGNYLFASGTKLTVL

SEQ ID NO: 30 - protein, 110 aa, synthetic construct
QSVLTQPPSASGTPGQRVTISCSGSSSNIGSNTVNWSRQLPGTAPKLLIYSNNQRPSGVPDRFSGSKSGTSASLAISGLQSEDEADYYCAAWDDSLNVYVFGTGTKVTVL

SEQ ID NO: 31 - protein, 107 aa, synthetic construct
KIVLTQSPSSLSASVGDTVTIACRASRDIRNDLAWYQQKPGKAPKLLIYGASSLQSGVPSRFSGSGSGTEFILTISSLQPEDFATYYCHRLNSYPLTFGGGTKVDIK

SEQ ID NO: 32 - protein, 111 aa, synthetic construct
NFMLTQPASLSGSPGQSITISCTGTSSDVGGYNYVSWYQQHPGYAPKLMIYDVSNRPSGVSNRFSGSKSGNTASLTISGLQAEDEADYYCSSYTSSSTPWVFGGGTKLTVL

SEQ ID NO: 33 - protein, 106 aa, synthetic construct
SYVLTQDPAVSVALGQTVRITCQGDSLRSYYASWYQQKPGQAPVLVIYGKNNRPSGIPDRFSGSSSGNTASLTITGAQAEDEADYYCNSRDSSGNQFGGGTKLTVL

SEQ ID NO: 34 - protein, 108 aa, synthetic construct
SSELTQDPAVSVALGQTVRITCQGDSLRTYYASWYQQRPGQAPILVLYGKNNRPSGIPDRFSGSSSGNTASLTITGAQAEDEADYYCNSRDSSGNHVVFGGGTKLTVL

SEQ ID NO: 35 - protein, 110 aa, synthetic construct
QSVLTQPASVSGSPGQSITISCTGTGSDVGSYNYVSWYQQNPGKAPKLMIYEVSNRPSGVSNRFSGSKSGNTASLTISGLQAEDEADYYCSSYTTSSTLVFGGGTKVTVL

SEQ ID NO: 36 - protein, 107 aa, synthetic construct
SELTQDPAVSVALGQTVRITCQGDSLRSYYASWYQQKPGQAPVLVIYGENSRPSGIPDRFSGSSSGNTASLTITGAQAEDEADYYCNSWDSSGNHVVFGGGTKLTVL

SEQ ID NO: 37 - protein, 107 aa, synthetic construct
AIRMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKLEIK

SEQ ID NO: 38 - protein, 111 aa, synthetic construct
DIVMTQSPLSLPVTPGEPASISCRSSQSLLHSNGYDYLDWYLQKPGQSPQLLIYLGSNRASGVPDRFSGSGSGTDFTLKISRVETEDVGIYYCMQGLQTPSFGQGTKLEIK

SEQ ID NO: 39 - protein, 109 aa, synthetic construct
SSELTQDPAVSVALGQTVRITCQGDSLRSYYASWYQQKPGQAPVPVIYGKNNRPSGIPDRFSGSSSGNTASLTITGAQAEDEADYYCNSRDSSSTHRGVFGGGTKLTVL

SEQ ID NO: 40 - protein, 107 aa, synthetic construct
DIQLTQSPSSLSASVGDRVTITCRASRSISTYLSWYQQKPGKAPKLLIYDASRLQNGVPSRFSGSGSDTDFTLTISSLQPEDFATYFCQQSYNPPWTFGQGTKLEIK

SEQ ID NO: 41 - protein, 111 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEAYYYCSSYTSSSDPWVFGGGTQLTVL

SEQ ID NO: 42 - protein, 107 aa, synthetic construct
NIQMTQSPSSLSASVGDRVTITCRAGQPISTYVNWYQHKPGKAPKLLIYGASNLQSGVPSRFSGGGSATDFTLTISSLQPEDFATYYCQQSYSSLLTFGDGTKVEIK

SEQ ID NO: 43 - protein, 15 aa, synthetic construct
GGGGSGGGGSGGGGS

SEQ ID NO: 44 - protein, 242 aa, synthetic construct
QVQLQESGGGLVKPGGPLRLSCAASGFTFSSYGMYWVRQAPGKGLEWVSTLSRSGSGTYYADSVKGRFTISRDNSKNTLYLQMNSLRAEDTAVYYCASIAVAGNYFDYWGQGTLVTVSSGGGGSGGGGSGGGGSSYVLTQDPAVSVALGQTVRITCQGDSLRSYYASWYQERPGQAPLLVIYGKNNRPSGIPDRFSGSNSGSTATLTISRVEAGDEGDYYCQVWDSINEQVVFGGGTKVTVL

SEQ ID NO: 45 - protein, 241 aa, synthetic construct
QVQLVQSGGGVVQPGRSLRLSCAATGIPFSGSGMHWVRQAPGKGLEWVTMIWYDGSNKFYADSVKGRFTISRDNSKNTLYLQMDSLRAEDTAVYFCARDKGVRSMDVWGLGTTVTVSSGGGGSGGGGSGGGGSNFMLTQPPSVSVAPGQTAKITCDGYSIRTKSVHWYQQKPGQAPVVVVHDDSDRPSGIPERFSGSNSGTTATLTISRVEAGDEADYYCQAWDSISEEVVFGGGTKLTVL

SEQ ID NO: 46 - protein, 240 aa, synthetic construct
QVQLQESGGGLVQPGGSLRLSCSASGFTFGTYAMRWVRQTSGKGLEWVSGIGVSGDAYYTDSVRGRFTISRDNSKNTLYLQMNTLRAEDTATYYCTRKSSTTSNDYWGRGTLVTVSSGGGGSGGGGSGGGGSSYVLTQDPAVSVALGQTVRITCQGDNIGSKSVHWYQQKPGQAPVLVVYDDSDRPSGIPERFSGSNSGTTATLTISSVEAGDEADYYCQAWDSISEHVIFGGGTKVTVL

SEQ ID NO: 47 - protein, 241 aa, synthetic construct
QVQLQESGGGLVQPGGSLRLSCAASGFTFSSYAMHWVRQAPGKGLEWVAVISYDGSNKYYADSVKGRFTISRDNSKNTLYLQMNSLRAEDTAVYYCARFSSGWYYFDYWGQGTLVTVSSGGGGSGGGGSGGGGSDIQMTQSPSFLSASVGDRITITCRASHDISSYFAWYQQKPGKAPKPLIYAASTLQSGVPSRFSGSGSGTEFTLTISSLQPEDFATYYCQQLGSYPLTFGGGTKLEIK

SEQ ID NO: 48 - protein, 245 aa, synthetic construct
QVQLLQSGGGLVQPGGSLRLSCAASGFTFSSYAMSWVRQAPGKGLEWVSAISGSGGSTYYTDSVKGRFTISRDNSKNTLYLQMNSLRAEDTAVYYCAKSHDYGDYAGFDYWGQGTLVTVSSGGGGSGGGGSGGGGSHVILTQDPAVSVALGQTVRITCQGDSLKSYYASWYQQKPGQAPVLVIYGKNNRPSGIPDRFSGSSSGTTASLTITGAQAEDEADYYCHSRDSSGTHLRVFGGGTKLTVL

SEQ ID NO: 49 - protein, 244 aa, synthetic construct
QVQLQESGGGLVKPGGSLRLSCAASGFTFSNAWMNWVRQAPGKGLEWVGRIKSKTDEGTTDYAAPVKGRFSISRDDSKNTLYLQMNSLKTEDTGVYYCTATKGLGGSKLGQGTLVTVSSGGGGSGGGGSGGGGSQSVLTQPPSASGTPGQRVTISCSGSSSNIGNNTVNWSRQLPGTAPKLLIYSNDQRPSGVPDRFSGSKSGTSASLAITGLQPEDEADYYCGTWDSSLSAYVFGTGTKLTVL

SEQ ID NO: 50 - protein, 245 aa, synthetic construct
QVQLVESGGGVVQPGRSLRLSCAASGFTFSSFGMHWVRRAPGKGLEWVAVISYDGSNQYYADSVKGRFTISRDNSKNTLYLQMNSLRAEDTAVYYCGSRPGGGYASGSTVAYWGQGTPVTVSSGGGGSGGGGSGGGGSSSELTQDPAVSVALGQTVRITCQGDSLRSYYASWYQQKPGQAPLLVIYGQNIRPSGIPDRFSGSSSGNSASLTITGAQAEDEADYYCHSRDSSGKYVFGVGTKVTVL

SEQ ID NO: 51 - protein, 237 aa, synthetic construct
QVQLVESGGGLVQPGGSLRLSCAASGFTFSSYAMGWVRQAPGKGLEWVSAISGSGGSTYYADSVKGRFTISRDNSKDTLYLQMNSLRAEDTAVYYCASRSLLDYWGQGTLVTVSSGGGGSGGGGSGGGGSNFMLTQDPAVSVALGQTVRITCQGDSLRSYYASWYQQKPGQAPLLVIYGKNNRPSGIPDRFSGSSSGNTASLTITGAQAEDEADYYCNSRDSSGNPVFGGGTKVTVL

SEQ ID NO: 52 - protein, 237 aa, synthetic construct
QVQLVESGGGLVQPGGSLRLSCAASGFTFSSYAMSWVRQAPGKGLEWVSAISGSGGSTYYADSVKGRFTISRDNSKDTLYLQMNSLRAEDTAVYYCASRSLLDYWGQGTLVTVSSGGGGSGGGGSGGGGSNFMLTQDPAVSVALGQTVRITCQGDSLRSYYASWYQQKPGQAPLLVIYGKNNRPSGIPDRFSGSSSGNTASLTITGAQAEDEADYYCNSRDSSGNPVFGGGTKVTVL

SEQ ID NO: 53 - protein, 243 aa, synthetic construct
QVQLQESGGGLVQLGGSLRLSCAASGFTFSSYAMSWVRQAPGKGLEWVSAISGSGGSTYYADSVKGRFTISRDNSKNTLYLQMSSLRAEDTAFYYCANSAYTGGWYDYWGHGTLVTVSSGGGGSGGGGSGGGGSSSELTQDPAVSVALGQTVKITCQGDSLRTYYASWYQQRPGQAPVLVIYGENSRPSGIPDRFSGSSSGNTASLTITGAQAEDEADYYCNSRDSSGNHLRVFGGGTKLTVL

SEQ ID NO: 54 - protein, 238 aa, synthetic construct
QVNLRESGGGLVQPGGFLRLSCAAFGFTFSGYWMSWVHPAPGKGLEWVANIKQDGSEKFYVDSVKGRFTISRDNAKNSLFLQMNSLRAEDTAVYFCARGLLSDYWGQGTLVPVSSGGGGSGGGGSGGGGSNFMLTQPPSVSVAPGKTASLTCGGYNIGTKSVHWYQQKPGQAPVVVVHDDSDRPSGIPERFSGSNSGTTATLTIIRVEAGDEADYYCQAWDSISEEVVFGGGTKLTVL

SEQ ID NO: 55 - protein, 242 aa, synthetic construct
QVQLQESGGGLVKPGGPLRLSCAASGFTFSSYGMYWVRQAPGKGLEWVSTLSRSGSGTYYAESVKGRFTISRDNSKNTLYFQMNSLRAEDTAVYYCASIAVAGNYFEYWGQGTLVTVSSGGGGSGGGGSGGGGSSYVLTQDPAVSVALGQTVRITCQGDSLRSYYASWYQERPGQAPLLVIYGKNNRPSGIPDRFSGSNSGSTATLTISRVEAGDEGDYYCQVWDSINEQVVFGGGTKVTVL

SEQ ID NO: 56 - protein, 243 aa, synthetic construct
QVQLQESGGGLVKPGESLRLSCAASGFTFSDHYMDWVRQAPGKGLEWVAYIRYDGSTKYYADSVKGRFTISRDNSKNTLYLQMNSLRPEDTAFYYCARLIAEAEGWFDPWGQGTLVTVSSGGGGSGGGGSGGGGSNFMLTQPPSVSVAPGKTARITCGGNNIGSKSVYWYQQKPGQAPVLVVYDDSDRPSGIPERFSGSNSGNTATLTISRVEAGDEADYYCQVWDSSSDHVVFGGGTKVTVL

SEQ ID NO: 57 - protein, 241 aa, synthetic construct
QVQLLQSAGGLVQPGGSLRLSCAASGFTFSTYAMNWVRQAPGKGLEWVSGISGSGGSTNYADSVKGRFTISRDSSKNTLFLQMNSLRAEDTAVYYCAKDYGSGWYDYWGQGTLVTVSSGGGGSGGGGSGGGGSSSELTQDPAVSVALGQTVRITCQGDSLRSYYASWYQERPGQAPLLVIYGRNERPSGIPDRFSASSSGNTASLTITGAQAEDEADYYCQVWDSFNEQVVFGGGTKLTVL

SEQ ID NO: 58 - protein, 245 aa, synthetic construct
QVQLVESGGGLVQPGGSLRLSCAASGFTFSSYAMSWVHQAPGKGLEWVSAISGSGGSTYYADSVKGRFTISRDNSKNTLYLQMNSLRAEDTAVYYCAKTYYGFWSGYYDYLGQGTLVTVSSGGGGSGGGGSGGGGSSSELTQDPAVSVGLGQTVTITCQGDSLRSYYANWYQQKPGQAPILVIYGENNRPSGIPDRFSGSSSGNTASLTITGAQAEDEADYYCHSRDSSGTHLRVFGGGTKLTVL

SEQ ID NO: 59 - protein, 244 aa, synthetic construct
QVQLLESGGGVVQPGTSLRLSCAASGFTFSNYAINWVRQAAGKGLEWVSGISGSGVSTSYADSVKGRFTVSRDNSKNTLYLQMNSLRVEDTALYYCAKNGGGPEYLQHWGQGTLVTVSSGGGGSGGGGSGGGGSQSVLTQPPSASGTPGQRVTISCSGSSSNIGNNTVNWSRQLPGTAPKLLIYSNDQRPSGVPDRFSGSKSGTSASLAITGLQPEDEADYYCGTWDSSLSAYVFGTGTKLTVL

SEQ ID NO: 60 - protein, 238 aa, synthetic construct
QVQLQESGGTLVQPGGSLRLSCAASGFTFSSYAMSWVRQAPGRGLEWVSTISGSGGSTYYADSVKGRFTISRDNSKNTLYLQMNSLRAEDTAVYYCAKGAYSGSYWGQGTLVTVSSGGGGSGGGGSGGGGSSSELTQDPAVSVALGQTVRITCQGDSLRSYYASWYQQKPGQAPSLVIYGENSRPSGIPDRFSGSSSGNTASLTITGAQAENEADYYCQAWDSSTAVVFGGGTKLTVL

SEQ ID NO: 61 - protein, 238 aa, synthetic construct
QVQLQESGGTLVQPGGSLRLSCAASGFTFSSYAMSWVRQAPGRGLEWVSTISGSGGSTYYADSVKGRFTISRDNSKNTLYLQMNSLRAEDTAVYYCAKGAYSGSHWGQGTLVTVSSGGGGSGGGGSGGGGSSSELTQDPAVSVALGQTVRITCQGDSLRSYYASWYQQKPGQAPSLVIYGENSRPSGIPDRFSGSSSGNTASLTITGAQAENEADYYCQAWDSSTAVVFGGGTKLTVL

SEQ ID NO: 62 - protein, 244 aa, synthetic construct
QVQLVESGAEVKKPGASVKVSCKASGYTFTSYGISWVRQAPGQGLEWMGWISAYNGNTNYAQKLQGRVTMTTDTSTSTAYMELRSLRSDDTAVYYCARPIYDSSGYDAFDIWGQGTMVTVSSGGGGSGGGGSGGGGSDIVMTQSPSTLSASIGDRVTITCRASEGIYHWLAWYQQKPGKAPKLLIYKASSLASGAPSRFSGSGSGTDFTLTISSLQPDDFATYYCQQYHTISRTFGPGTKVDIK

SEQ ID NO: 63 - protein, 244 aa, synthetic construct
QVQLVESGGGLVKPGGSLRLSCAASGFTFSSYAMHWVRQAPGKGLEWVAVISYDGSNKYYADSVKGRFTISRDNSKNTLYLQMNSLRAEDTAVYFCVRPSDSGWSFEHWGQGTLVPVSSGGGGSGGGGSGGGGSQSVLTQPPSASGTPGQRVTISCSGSSSNIGNNTVNWSRQLPGTAPKLLIYSNDQRPSGVPDRFSGSKSGTSASLAITGLQPEDEADYYCGTWDSSLSAYVFGTGTKLTVL

SEQ ID NO: 64 - protein, 245 aa, synthetic construct
QVQLQESGGGLVQPGGSLRLSCAASGFTFSSYAMSWVRQAPGKGLEWVAVISYDGSNKYYADSVKGRFTISRDNSKNTLYLQMNSLRAEDTAVYYCVRGDRSYGAEYFQHWGQGTLVTVSSGGGGSGGGGSGGGGSSSELTQDPAVSVASGQTVRITCQGDSLRSYYASWYQQKPGQAPLLVIYGKNIRPSGIPDRFSGSTSGNSASLTITGAQAEDEADYYCNSRDSSGNRNWVFGGGTKLTVL

SEQ ID NO: 65 - protein, 246 aa, synthetic construct
QVQLQESGGGLVKPGGSLRLSCAASGFTSSSYAMHWVRQAPGKGLEYVSAIGGNGGTYYADSVKGRFTISRDNSKNTLYLQMNSLRAEDTAVYYCAKEGEQWLEYRYYYGMDVWGQGTTVTVSSGGGGSGGGGSGGGGSSSELTQDPAVSVALGQTVRITCQGDSLRSYYASWYQQKPGQAPSLVIYGENSRPSGIPDRFSGSSSGNTASLTITGAQAENEADYYCQAWDSSTAVVFGGGTKLTVL

SEQ ID NO: 66 - protein, 250 aa, synthetic construct
QVQLVESGGGVVQPGRSLRLSCTASGFTFSSYGMHWVRQAPGKGLEWVAVISYDGSNKYYADSVKGRFTISRDNSKNTLYLQMNSLRAEDTAVYYCARGGRYSSNWFSYYYYGMDVWGQGTTVTVSSGGGGSGGGGSGGGGSNFMLTQPPSVSVAPGKTARITCGGNNIGSKSVYWYQQKPGQAPVLVVYDDSDRPSGIPERFSGSNSGNTATLTISRVEAGDEADYYCQVWDSSSDHVVFGGGTKVTVL

SEQ ID NO: 67 - protein, 15 aa, synthetic construct
GGGGSGGGGSGGGGS

SEQ ID NO: 68 - protein, 10 aa, synthetic construct
GGGGSGGGGS

SEQ ID NO: 69 - protein, 5 aa, synthetic construct
GGGGS

SEQ ID NO: 70 - protein, 20 aa, synthetic construct
GSGGGGSGGGGSGGSGGGGS

SEQ ID NO: 71 - protein, 6 aa, synthetic construct
SGGGGS

SEQ ID NO: 72 - protein, 4 aa, synthetic construct
GGGS

SEQ ID NO: 73 - protein, 4 aa, synthetic construct
VPGV

SEQ ID NO: 74 - protein, 5 aa, synthetic construct
VPGVG

SEQ ID NO: 75 - protein, 6 aa, synthetic construct
GVPGVG

SEQ ID NO: 76 - protein, 8 aa, synthetic construct
GVGVPGVG

SEQ ID NO: 77 - protein, 10 aa, synthetic construct
VPGVGVPGVG

SEQ ID NO: 78 - protein, 7 aa, synthetic construct
GGSSRSS

SEQ ID NO: 79 - protein, 18 aa, synthetic construct
GGSSRSSSSGGGGSGGGG

SEQ ID NO: 80 - protein, 8 aa, synthetic construct
GLTVNNYA

SEQ ID NO: 81 - protein, 8 aa, synthetic construct
ISYDGNNK

SEQ ID NO: 82 - protein, 9 aa, synthetic construct
AKGGGYFDL

SEQ ID NO: 83 - protein, 9 aa, synthetic construct
SSNIGAGYD

SEQ ID NO: 84 - skipped

SEQ ID NO: 85 - protein, 9 aa, synthetic construct
SSYTSGTWL

SEQ ID NO: 86 - protein, 116 aa, synthetic construct; identical to SEQ ID NO: 1

SEQ ID NO: 87 - protein, 109 aa, synthetic construct; identical to SEQ ID NO: 22

SEQ ID NO: 88 - protein, 446 aa, synthetic construct
QVQLVQSGGGVVQPGRSLRLACAASGLTVNNYAMHWVRQAPGKGLEWVAVISYDGNNKYYADSVKGRFTISRDNSKNTLYLQMNSLRAEDTAVYYCAKGGGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 215 aa, synthetic construct
QSVLTQPPSVSGAPGQRVTISCTGSSSNIGAGYDVHWYQQLPGTAPKLLIYGNNNRPSGVPDRFSGSKSGTSASLAITGLQAEDEADYYCSSYTSGTWLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2026/0355364 A1
(43) Pub. Date: Nov. 12, 2026

(54) RADIOIMMUNOCONJUGATES AND THERAPEUTIC USES THEREOF

(71) Applicant: The Regents of the University of California, Oakland, CA (US)

(72) Inventors: Kondapa Naidu Bobba, San Francisco, CA (US); Robert Flavell, San Francisco, CA (US); Bin Liu, San Francisco, CA (US); Scott Bidlingmaier, San Francisco, CA (US); Anil Bidkar, San Francisco, CA (US)

(73) Assignee: The Regents of the University of California, Oakland, CA (US)

(21) Appl. No.: 19/669,823

(22) Filed: May 6, 2026

Related U.S. Application Data
(63) Continuation of application No. 18/867,159, filed on May 22, 2023.
(60) Provisional application No. 63/344,537, filed on May 20, 2022.

(51) Int. Cl. A61K 51/10; C07K 16/28

(52) U.S. Cl. CPC A61K 51/1027; A61K 51/1093; C07K 16/2896; A61K 2121/00

(57) ABSTRACT

Provided are radioimmunoconjugates and methods of using the radioimmunoconjugates for treating cancer and for detecting tumor cells.

### CROSS-REFERENCES TO RELATED APPLICATIONS

The present application is a continuation of U.S. patent application Ser. No. 18/867,159, filed Nov. 19, 2024, which is a U.S. National Stage of PCT/US2023/023157, international filing date May 22, 2023, which claims priority benefit of U.S. Provisional Application No. 63/344,537, filed May 20, 2022, the disclosures of which are herein incorporated by reference in their entirety for all purposes.

### STATEMENT AS TO RIGHTS TO INVENTIONS MADE UNDER FEDERALLY SPONSORED RESEARCH AND DEVELOPMENT

This invention was made with government support under W81XWH-21-1-0792 awarded by the Defense Health Agency, Medical Research and Development Branch, and R01 CA279203 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED ON A COMPACT DISK

The instant application contains a Sequence Listing which has been submitted electronically in XML format and is hereby incorporated by reference in its entirety. Said XML copy, created on Dec. 4, 2024, is named 081906-1473819-248610US_SL.xml and is 88,154 bytes in size.

### BACKGROUND OF THE INVENTION

Targeted radionuclide therapy has been demonstrated as an outstanding methodology for late-stage cancer treatments where therapeutic alternatives are limited (Gudkov, S. V. et al. (2016) Int. J Mol. Sci. 17(33), 1-19. Although few α-radionuclides are suitable for treatments, Actinium-225 (²²⁵Ac) with a long half-life of 10 days (see, FIG. 1) has attracted great attention due to its unique properties such as “nanogenerator’ status and produces a total of 4 α and 2 β—particles in its decay chain (McDevitt, M. R. et al. (2001) Science, 294(5546), 1537-40). However, its clinical translation is restricted by, for example, (1) toxicities associated with the irradiation of healthy tissues following target engagement (e.g., xerostomia after ²²⁵Ac-PSMA-617 therapy), (2) the non-specific accumulation of the radiopharmaceutical (off-target toxicities) (e.g., clearance organs), (3) lack of chelators optimized for ²²⁵Ac, and (4) lack of suitable radiolabeling methods. Thus, there is an urgent and unmet need for new strategies that minimize the inherent toxicity of ²²⁵Ac-based radioimmunotherapy and maximize the anti-tumor efficacy of the targeting agents.

### BRIEF SUMMARY OF THE INVENTION

The present invention provides radioimmunoconjugates comprising an antibody that specifically binds to CD46; a radionuclide; and a chelator, wherein the chelator chelates the radionuclide, and wherein the chelator is coupled to the antibody through a linker comprising poly(ethylene glycol), i.e., a (PEG)ₙ-linker, wherein n is 4, 6, 8, or 12. Such immunoconjugates are useful for treating cancer and for detecting tumor cells.

In one aspect, provided is an immunoconjugate having a structure according to Formula I:

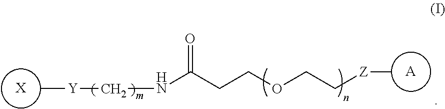

In Formula I, X is a chelator moiety; Y is selected from the group consisting of —O— and —NR—; Z is a moiety selected from the group consisting of:

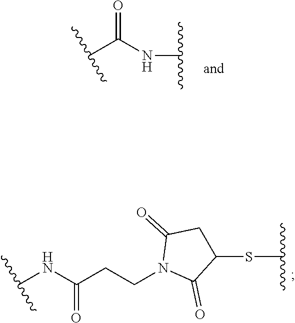 [SMILES: *NC(*)=O ; *NC(=O)CCN1C(=O)CC(S*)C1=O]

A is an antibody that specifically binds to CD46; subscript m is 3 or 5; subscript n is 4, 6, 8, 10, 12, 14, or 16; and R selected from the group consisting of H, OH, and a negative charge.

In some embodiments, the immunoconjugate has a structure according to Formula Ia as follows:

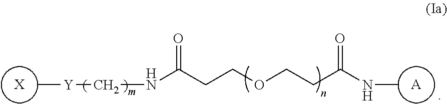

In other embodiments, the immunoconjugate has a structure according to Formula Ib as follows:

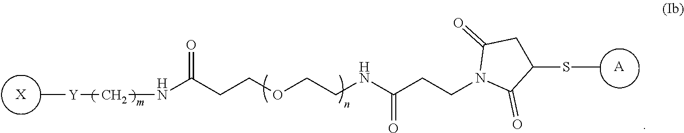

In some embodiments, the chelator moiety “X” includes, but is not limited to, one of the following structures:

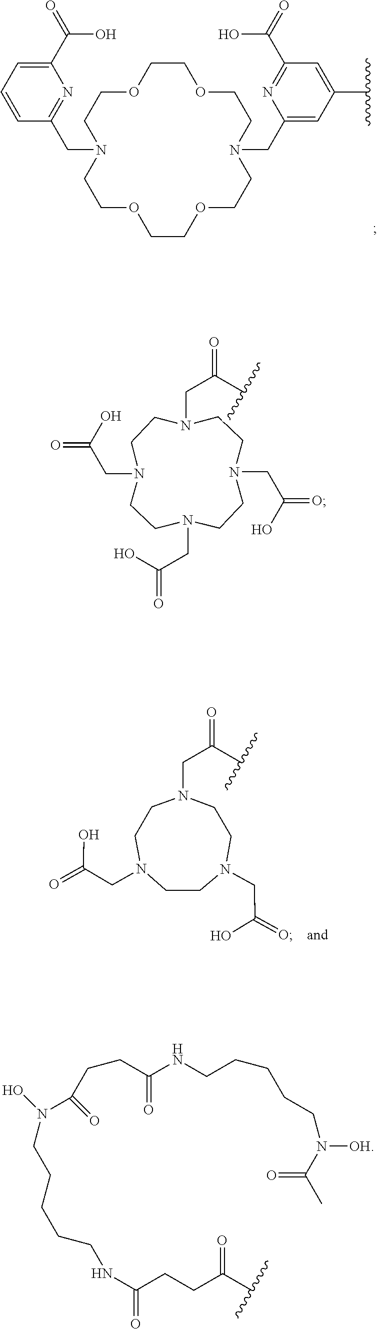 [SMILES: *c1cc(CN2CCOCCOCCN(Cc3cccc(C(=O)O)n3)CCOCCOCC2)nc(C(=O)O)c1 ; *C(=O)CN1CCN(CC(=O)O)CCN(CC(=O)O)CCN(CC(=O)O)CC1 ; *C(=O)CN1CCN(CC(=O)O)CCN(CC(=O)O)CC1 ; *C(=O)CCC(=O)NCCCCCN(O)C(=O)CCC(=O)NCCCCCN(O)C(C)=O]

In one embodiment of the immunoconjugate of Formula I, X is a chelator moiety having the following structure:

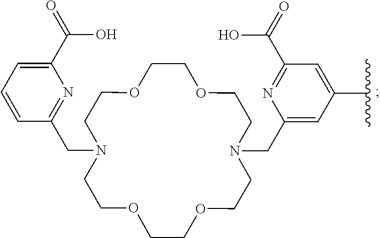 [SMILES: *c1cc(CN2CCOCCOCCN(Cc3cccc(C(=O)O)n3)CCOCCOCC2)nc(C(=O)O)c1]

Y is —O—; and subscript m is 3. In this embodiment, n is 4, 6, 8, or 12. In other embodiments, n is 4 or 8.

In another embodiment of the immunoconjugate of Formula I, X is a chelator moiety having the following structure:

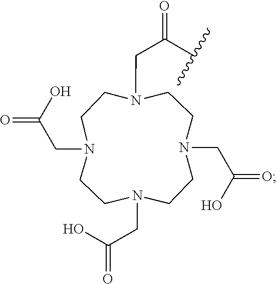 [SMILES: *C(=O)CN1CCN(CC(=O)O)CCN(CC(=O)O)CCN(CC(=O)O)CC1]

Y is —NR—; and subscript m is 3. In this embodiment, n is 4, 6, 8, or 12. In other embodiments, n is 4 or 8.

In another embodiment of the immunoconjugate of Formula I, X is a chelator moiety having the following structure:

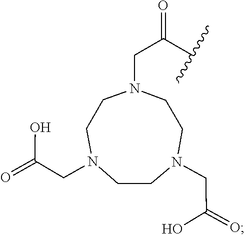 [SMILES: *C(=O)CN1CCN(CC(=O)O)CCN(CC(=O)O)CC1]

Y is —NR—; and subscript m is 3. In this embodiment, n is 4, 6, 8, or 12. In other embodiments, n is 4 or 8.

In another embodiment of the immunoconjugate of Formula I, X is a chelator moiety having the following structure:

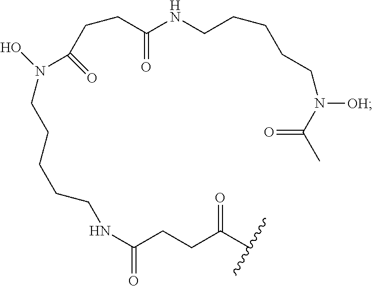 [SMILES: *C(=O)CCC(=O)NCCCCCN(O)C(=O)CCC(=O)NCCCCCN(O)C(C)=O]

Y is —NR—; and subscript m is 5. In this embodiment, n is 4, 6, 8, or 12. In other embodiments, n is 4 or 8.

In another aspect, radioimmunoconjugates are provided, the radioimmunoconjugate comprising an immunoconjugate of Formula I, and an alpha-emitting radionuclide, wherein the chelator moiety of the immunoconjugate of Formula I chelates the alpha-emitting radionuclide. Exemplary alpha-emitting radionuclide suitable for use in the radioimmunoconjugates described herein include, but are not limited to, ²²⁵Ac, ²¹³Bi, ²²⁴Ra, ²¹²Pb, ²²⁷Th, ²²³Ra, ²¹¹At, and ¹⁴⁹T. In some embodiments, the radionuclide is ²²⁵Ac.

In one embodiment, the radioimmunoconjugate has one of the following structures:

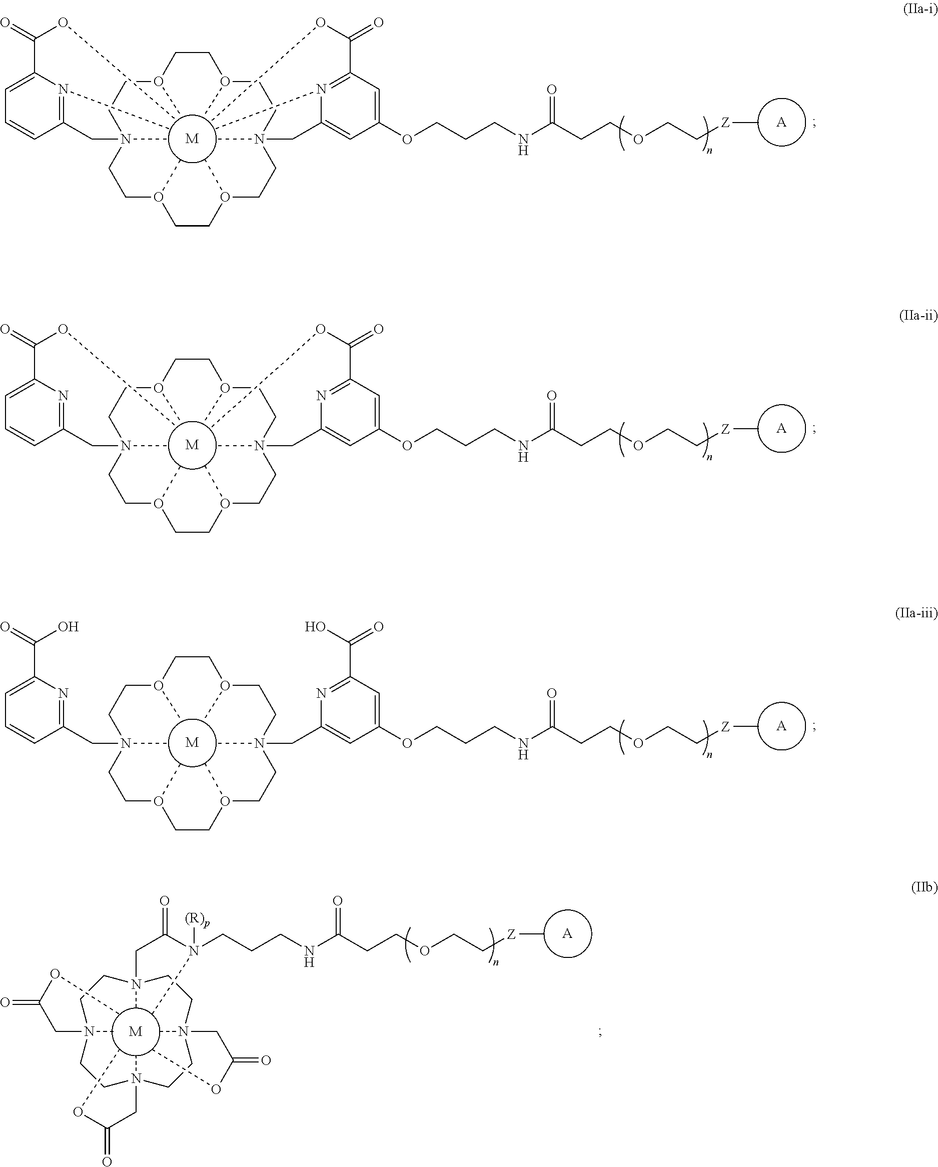

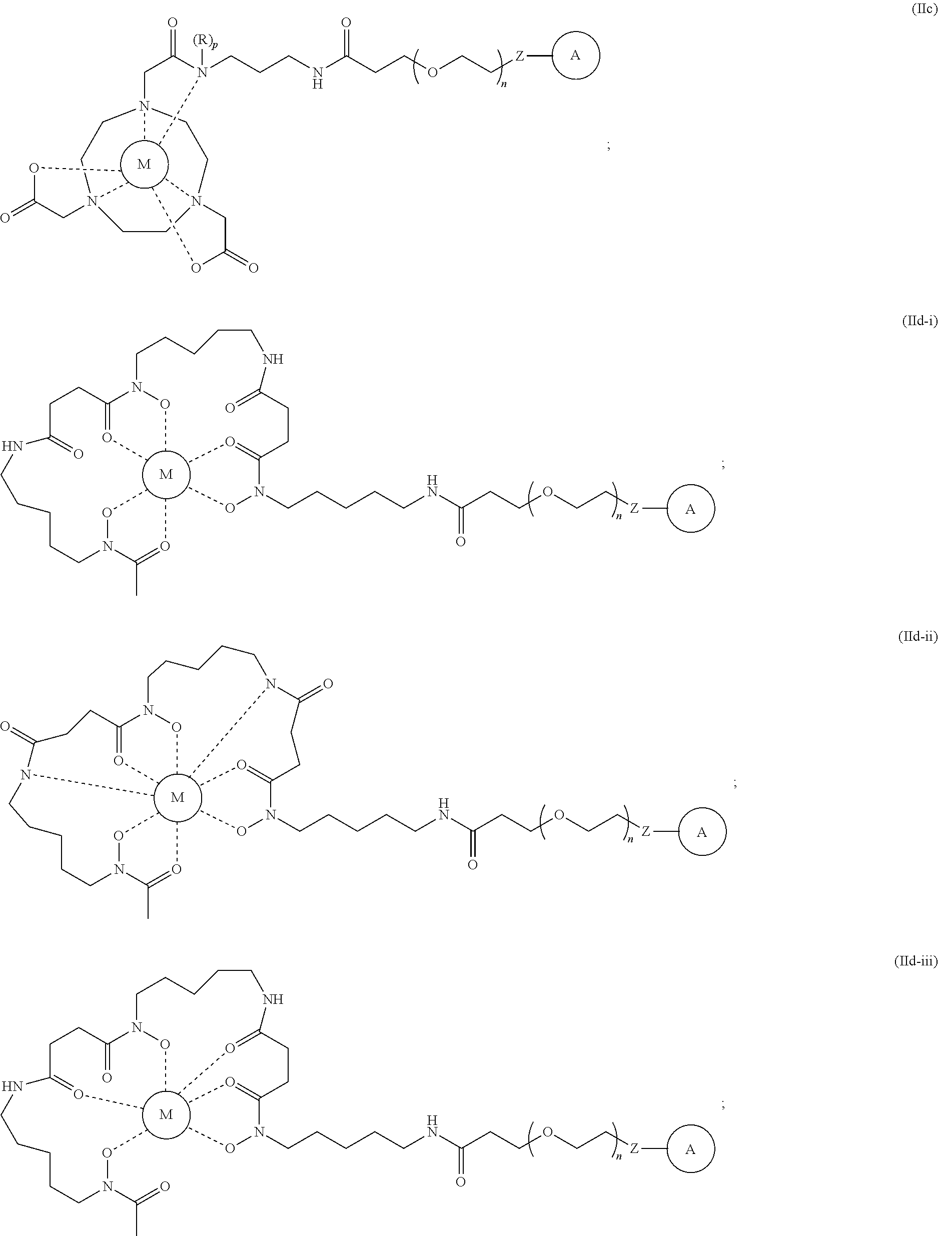

In the above Formulae (IIa-i) through (IId-iii), M is an alpha-emitting radionuclide, such as ²²⁵Ac, and subscript p, when present, is 0 or 1.

In some embodiments of the immunoconjugate of Formula I and the corresponding radioimmunoconjugates, the antibody or “A” is the antibody YS5. In other embodiments, the antibody or “A” comprises heavy chain CDRs 1, 2 and 3 and light chain CDRs 1, 2, and 3 of any one of YS5, YS5F, YS5v1D, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, or UA8kappa. In other embodiments, the antibody or “A” comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively.

In another aspect, provided are pharmaceutical compositions comprising an immunoconjugate of Formula (I), (Ia) or (Ib), or a radioimmunoconjugate of Formulae (IIa), (IIa-i), (IIa-ii), (IIa-iii), (IIb), (IIc), (IId), (IId-i), (IId-ii), or (IId-iii), and a pharmaceutically acceptable excipient.

In another aspect, a method of treating cancer is provided, the method comprising administering to the subject an radioimmunoconjugate, the radioimmunoconjugate comprising an immunoconjugate of Formula and an alpha-emitting radionuclide, wherein the chelator moiety of the immunoconjugate of Formula I chelates the alpha-emitting radionuclide. In one embodiment, the alpha-emitting radionuclide is ²²⁵Ac.

In one embodiment, the cancer is a CD46 expressing cancer. CD46 expressing cancers include, but are not limited to, ovarian cancer, breast cancer, lymphoma, hepatocellular carcinoma, lung cancer, prostate cancer, and colon cancer. In one embodiment, the CD46 expressing cancer is prostate cancer. In one embodiment, the radioimmunoconjugate has the structure according to Formula IIa:

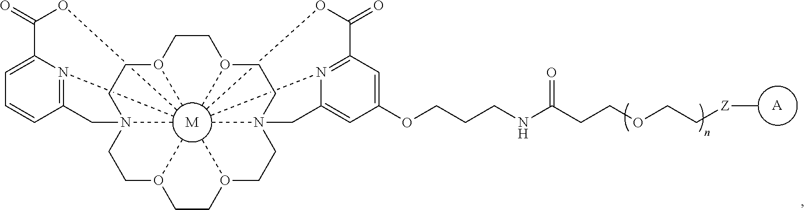

wherein M is the alpha-emitting radionuclide.

In one aspect, a method of treating prostate cancer in a subject is provided, the method comprising administering to the subject an radioimmunoconjugate, wherein the immunoconjugate of Formula I and an alpha-emitting radionuclide, wherein the chelator moiety of the immunoconjugate of Formula I chelates the alpha-emitting radionuclide. In one embodiment, the alpha-emitting radionuclide is ²²⁵Ac. In one embodiment, the radioimmunoconjugate has the structure according to Formula IIa:

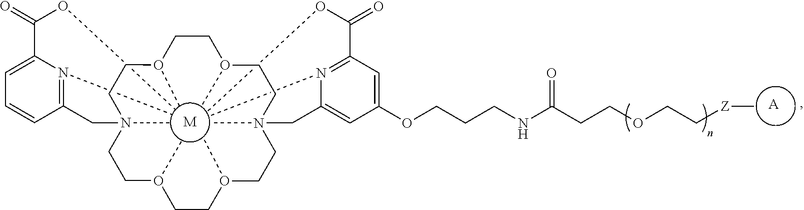

wherein M is the alpha-emitting radionuclide.

### BRIEF DESCRIPTION OF THE DRAWINGS

FIG. 1 illustrates the decay scheme for Actinium-225.

FIG. 2 illustrates the magnetic bead based radioligand binding assay for various ratios of ²²⁵Ac-Macropa per YS5 IgG (#).

FIG. 3 illustrates (i) the biodistribution of ²²⁵Ac-Macropa-PEG_{0,4&8}-YS5 conjugates in 22Rv1 xenografts at different time points on Day 1, 2, 4 & 7 (3A); (ii) tumor uptake at different time points in 22Rv1 xenografts for the indicated radiopharmaceuticals (n=4 for most agents, n=3 for ²²⁵Ac-DOTA-YS5 (3B) [(#)=chelate to Abratio]; and (iii) the Tumor to Liver ratios (3C).

FIG. 4 illustrates (A) Bioconjugation of YS5 with Macropa-NCS and Macropa-PEG4,8-TFP ester followed by radiolabeling with 225Ac(NO3)3; (B) Structures of Macropa-PEG0,4,8-YS5; (C) Radiochemical yields for 225Ac-Macropa-PEG0(0.55)-YS5, 225Ac-Macropa-PEG4(0.91)-YS5, 225Ac-Macropa-PEG8(0.96)-YS5 and 225Ac-DOTA(8.7)-YS5; (#) denotes number of chelators per YS5 antibody.

FIG. 5 illustrates SEC HPLC (A) and stability studies (B) of ²²⁵Ac-Macropa-PEG₀(0.55)-YS5, ²²⁵Ac-Macropa-PEG₄(0.91)-YS5, and ²²⁵Ac-Macropa-PEG₈(0.96)-YS5 [from left to right]

FIG. 6 illustrates tumor uptake for conjugates PEG₀(4.5), PEG₀(0.55), PEG₄(0.91), PEG₈(0.96), PEG₈(7.7) and DOTA (8.7) at various time points 1 d, 2 d, 4 d and 7 d in 22Rv1 xenografts (A) and statistical analysis PEG4(0.91) Vs PEG₀(4.5), PEG₀(0.55), PEG₈(0.96), PEG₈(7.7) and DOTA (8.7) (B).

FIG. 7 illustrates the biodistribution of nearly 1:1 ratios of 225Ac-Macropa-PEG0,4,8-YS5, and DOTA-YS5 in 22Rv1 xenografts at different time points day 1 (A), day 2 (B), day 4 (C), day 7 (D).

FIG. 8 illustrates tumor to blood (A), Muscle (B), Liver (C), and Kidney (D) ratios at various time points 1 d, 2 d, 4 d and 7 d in 22Rv1 xenografts.

FIG. 9 illustrates an exemplary treatment study plan.

FIG. 10 illustrates the antitumor activity of ²²⁵Ac-Macropa-PEG4(0.91)-YS5 and ²²⁵Ac-DOTA(8.7)-YS5 in subcutaneous 22Rv1 xenografts with 0.5 μCi, 0.25 μCi, and 0.125 μCi doses.

FIG. 11 illustrates an exemplary fractionated dose treatment study plan.

FIG. 12 illustrates antitumor activity of ²²⁵Ac-Macropa-PEG4(0.91)-YS5 and ²²⁵Ac-DOTA(8.7)-YS5 in subcutaneous 22Rv1 xenografts with 0.5 μCi, 0.25 μCi, and 0.125 μCi doses.

### DEFINITIONS

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described. In this application, the use of the singular includes the plural unless specifically stated otherwise. It is noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting.

As used herein, ranges and amounts can be expressed as “about” a particular value or range. About also includes the exact amount, but also allows a reasonable amount of deviation of the modified term such that the end result is not significantly changed. The term about should be construed as including a deviation of at least ±5% of the modified term if this deviation would not negate the meaning of the word it modifies. Generally, the term “about” includes an amount that would be expected to be within experimental error.

The terms “antibody” and “immunoglobulin” are used interchangeably herein and are used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The terms “monoclonal antibody” and “mAb” are used interchangeably herein and refer to an antibody obtained from a substantially homogeneous population of antibodies, i.e., the individual antibodies of the population are identical except for possible naturally occurring mutations that may be present in minor amounts.

The term “hypervariable region,” as used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest Fifth Edition, U.S. Department of Health and Human Services, NIH Publication No. 91-3242 (referred to herein as “Kabat et al”) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Chothia and Lesk, (1987) J. Mol. Biol., 196:901-917). “Framework” or “FR” residues are those variable domain residues other than the hypervariable region residues, as herein deemed.

In some instances, the CDRs of an antibody is determined according to (i) the Kabat numbering system Kabat et al. (1991) Sequences of Proteins of Immunological Interest Fifth Edition, U.S. Department of Health and Human Services, NIH Publication No. 91-3242; or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273: 927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

As used herein, the term “antigen-binding site” refers to the part of the antigen binding molecule that specifically binds to an antigenic determinant. More particularly, the term “antigen binding site” refers the part of an antibody that comprises the area which specifically binds to and is complementary to part or all of an antigen. Where an antigen is large, an antigen binding molecule may only bind to a particular part of the antigen, which part is termed an epitope. An antigen-binding site may be provided by, for example, one or more variable domains (also called variable regions). Preferably, an antigen-binding site comprises an antibody light chain variable region (VL) and an antibody heavy chain variable region (VH).

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, a molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10-7 M or less, e.g. from 10-⁷M to 10-¹³ M, e.g. from 10-⁹ M to 10-¹³ M).

As noted above, depending on the amino acid sequence of the constant domain of their heavy chains, immunoglobulins can be assigned to different classes. There are five major classes of human immunoglobulins: IgA, IgD, IgE, IgG, IgM, and IgY, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity. The light chains of antibodies (immunoglobulins) from any vertebrate species can be assigned to one of two clearly distinct types, called kappa (κ) and lambda (λ), based on the amino acid sequences of their constant domains.

The term “chimeric antibody,” as used herein refers to an antibody in which a portion of the heavy and/or light chain is derived from a particular source (e.g., protein) or species, while the remainder of the heavy and/or light chain is derived from a different source (e.g., protein) or species.

The term “recombinant human antibody,” as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. In some cases, the recombinant human antibodies have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The term “valent” as used herein denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule. The bispecific antibodies according to the invention are at least “bivalent” and may be “trivalent” or “multivalent” (e.g. “tetravalent” or “hexavalent”). In a particular aspect, the antibodies of the present invention have two or more binding sites and are bispecific. That is, the antibodies may be bispecific even in cases where there are more than two binding sites (i.e. that the antibody is trivalent or multivalent). In particular, the invention relates to bispecific bivalent antibodies, having one binding site for each antigen they specifically bind to.

The term “monospecific” antibody as used herein denotes an antibody that has one or more binding sites each of which bind to the same epitope of the same antigen.

The term “linker” as used herein means a chemical moiety comprising or derived from a group of atoms that is covalently attached to an antibody and that is also covalently attached to a chelator. The linker used in the immunoconjugates described herein comprises poly(ethylene glycol) (PEG). PEGs of varying chain lengths can be used in the linker that covalent attaches the antibody to the chelator. In some embodiments, the poly(ethylene glycol) portion of the linker is -(PEG)ₙ-, wherein n is 4, 6, 8, or 12. An exemplary linker comprising poly-ethylene glycol is a (PEG)_{4,6,8,12} linker with malemide and N-hydroxysuccinamide (NHS) functional groups (Mal-PEGₙ-NHS, wherein n is 4, 6, 8, 12).

The terms “individual(s)”, “subject(s)” and “patient(s)” are used interchangeably herein and refer to any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “cancer” and “tumor” are used interchangeably herein, encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules described herein are used to delay development of a disease or to slow the progression of a disease.

### DETAILED DESCRIPTION OF THE INVENTION

### A. Introduction

Provided are radioimmunoconjugates that include an antibody that binds CD45, a radionuclide, a chelator that chelates the radionuclide and that is coupled to the antibody through a poly(ethylene glycol) (PEGₙ) linker. The radioimmunoconjugates described herein are useful for treating cancer, especially, CD46 expressing cancer (e.g., prostate cancer), and for detecting tumor cells. The radioimmunoconjugates described herein, which include short PEGylated macrocyclic chelator complexes, advantageously alter the biodistribution and therapeutic efficacy of radionuclide imaging and therapy. It has surprisingly been discovered that inserting short poly(ethyleneglycol) (PEG) linkers (PEG₄₋₁₂) into the disclosed radioimmunoconjugates promotes higher tumor uptake and, in turn, lowers the burden of unnecessary radiation on other major organs, such as the liver, spleen, etc. Thus, the radioimmunoconjugates of the present invention improve treatment efficacy by lowering the unnecessary radiation burden to the patient.

### B. Antibodies that Bind CD46

The radioimmunoconjugates of the present invention include an antibody that specifically binds to CD46. In some embodiments, the anti-CD46 antibody is an internalizing antibody, meaning that the antibodies are internalized by tumor cells, for example via the macropinocytosis pathway. For example, the antibodies can be internalized by the tumor-selective macropinocytosis pathway, without the need of crosslinking. and localize to the lysosomes, which makes them well suited for use as antibody drug conjugates (ADCs) and other targeted therapeutics that utilize intracellular payload release. A large number of anti-CD46 antibodies are known, including but not limited to those described in U.S. Pat. Nos. 9,593,162; 9,567,402 and 10,533,056.

In some embodiments, the anti-CD46 specifically bind CD46, in particular domains 1 and/or 2, and are internalized by multiple myeloma cells (and other CD46 positive cancer cells, such as those described herein) in situ, e.g., when the cancer cell is in the tissue microenvironment. As indicated above, such antibodies are useful for targeting CD46 expressing cancers.

The antibodies designated herein as YS5, YS5F, YS5v1D, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and UA8kappa (see, e.g., Table Error! Reference source not found.) are exemplary anti-CD46 antibodies. In certain embodiments, antibodies that comprise VL CDR1 and/or VL CDR2, and/or VL CDR3, and/or VH CDR1 and/or VH CDR2, and/or VH CDR3 of one or more of these antibodies are contemplated. In certain embodiments, antibodies that comprise the VH domain and/or the VL domain of one or more of these antibodies are contemplated. Also contemplated are antibodies that compete for binding at CD46 with one or more of as YS5, YS5F, YS5v1D, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and/or UA8kappa.

The amino acid sequences of the VH and VL domains of YS5, YS5F, YS5v1D, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and/or UA8kappa antibodies are shown in Table Error! Reference source not found.

YS5 and YS5F differ by one amino acid in VH CDR1 (L vs. F). YS5 and YS5v1D have identical VH but one amino acid difference in the VL CDR2 (N vs. D). 3G7HY, 3G7NY, 3G7RY (aka 3G8), and 3G7 have one residue difference in VH CDR3, but entirely different VLs. YS6 and 3G7 have identical VH but different VL.

TABLE 1
Novel human anti-CD46 antibody sequences.

| | VH | VL |
|---|---|---|
| YS5 | QVQLVQSGGGVVQPGRSLRLACAASGLTV | QSVLTQPPSVSGAPGQRVTISCTGSSSNIGA |
| | NNYAMHWVRQAPGKGLEWVAVISYDGNNK | GYDVHWYQQLPGTAPKLLIYGNNNRPSGVPD |
| | YYADSVKGRFTISRDNSKNTLYLQMNSLR | RFSGSKSGTSASLAITGLQAEDEADYYCSSY |
| | AEDTAVYYCAKGGGYFDLWGRGTLVTVSS | TSGTWLFGGGTKLTVL |
| | (SEQ ID NO: 1) | (SEQ ID NO: 22) |
| YS5F | QVQLVQSGGGVVQPGRSLRLACAASGFTV | QSVLTQPPSVSGAPGQRVTISCTGSSSNIGA |
| | NNYAMHWVRQAPGKGLEWVAVISYDGNNK | GYDVHWYQQLPGTAPKLLIYGNNNRPSGVPD |
| | YYADSVKGRFTISRDNSKNTLYLQMNSLR | RFSGSKSGTSASLAITGLQAEDEADYYCSSY |
| | AEDTAVYYCAKGGGYFDLWGRGTLVTVSS | TSGTWLFGGGTKLTVL |
| | (SEQ ID NO: 2) | (SEQ ID NO: 23) |
| YS5vlD | QVQLVQSGGGVVQPGRSLRLACAASGFTV | QSVLTQPPSVSGAPGQRVTISCTGSSSNIGA |
| | NNYAMHWVRQAPGKGLEWVAVISYDGNNK | GYDVHWYQQLPGTAPKLLIYGDNNRPSGVPD |
| | YYADSVKGRFTISRDNSKNTLYLQMNSLR | RFSGSKSGTSASLAITGLQAEDEADYYCSSY |
| | AEDTAVYYCAKGGGYFDLWGRGTLVTVSS | TSGTWLFGGGTKLTVL |
| | (SEQ ID NO: 3) | (SEQ ID NO: 24) |
| SB1HGNY | QVQLQQSGGGVVQPGRSLRLSCAASGFTF | DIQMTQSPSFLSASVGDRVTITCRASQGISS |
| | SSYAMHWVRQAPGKGLEWVAFIRSDGSKK | YLAWYQQKPGKAPKLLIYAASTLQSGVPSSF |
| | YYADSVKGRFTISRDNSKNTLYLQMNSLR | SGSGSGTEFTLTISSLQPEDFATYYCQQLAS |
| | AEDTAVYYCARHGNYFDSWGQGTLVTVSS | YPLTFGGGTKVDIK |
| | (SEQ ID NO : 4) | (SEQ ID NO: 25) |
| YS12 | QVQLVESGGGVVQPGRSLRLSCAASGFTF | SSELTQDPAVSVALGQTVRITCQGDSLRSYY |
| | STYGMHWVRQAPGKGLEWLSFISYDGDEK | VSWFQQKPGQAPVFVMYGQNNRPSGISERFS |
| | YYADSVKGRFTISRDNSKNTLYLQMNSLR | GSSSGNTASLIITGAQAEDEADYYCHSRDSS |
| | AEDTAVYWCAKASGYGMGILDYWGQGTLV | GTHLRVFGGGTKLTVL |
| | TVSS | (SEQ ID NO: 26) |
| | (SEQ ID NO: 5) | |
| 3G7RY | EVQLVESGGGLVQPGGSLRLSCAASGFTF | QSALTQPPSASATPGQRVTISCSGRTSNIGS |
| aka | SDYYMSWIRQAPGKGLEWVSYISSSGSTI | NHVYWYQQLPGTAPKLLIYRNNQRPSGVPDR |
| 3G8 | YYADSVKGRFTISRDNSKNTLYLQMNSLR | FSGSKSGTSASLAISGLRSEDEADYYCATWD |
| | AEDTAVYYCARDYGRIAAAGRRYWGQGTL | DSLSGEVFGGGTKLTVL |
| | VTVSS | (SEQ ID NO: 27) |
| | (SEQ ID NO: 6) | |
| YS6 | QVQLQESGGGVVRPGGSLRLSCAASGFTF | SSELTQDPAVSVALGQTVRITCQGDSLRSYY |
| | SDYYMSWIRQAPGKGLEWVSYISSSGSTI | ASWYQQKPGQAPVLVIYGKNNRPSGIPDRFS |
| | YYADSVKGRFTISRDNSKNTLYLQMNSLR | GSSSGNTASLTITGAQAEDEADYYCNSRDSS |
| | AEDTAVYYCARDYGRIAAAGRHYWGQGTL | GTHLEVFGGGTKVTVL |
| | VTVSS | (SEQ ID NO: 28) |
| | (SEQ ID NO: 7) | |
| YS1 | EVQLVESGGGLVQPGGSLRLSCAASGFTF | SSELTQDPAVSVALGQTVRITCQGDTLSTYY |
| | SDYYMSWIRQAPGKGLEWVSYISSSGSTI | ANWYQQKPGQAPVLVIYGKNNRPSGIPDRFS |
| | YYADSVKGRFTISRDNSKNTLYLQMNSLR | GSSSGNTASLTITGAQAEDEADYYCHSRDIS |
| | AEDTAVYYCARDYGRIAAAGRHYWGQGTL | GNYLFASGTKLTVL |
| | VTVSS | (SEQ ID NO: 29) |
| | (SEQ ID NO: 8) | |
| YS3 | QVQLQESGGGLVQPGGSERLSCAASGFTF | QSVLTQPPSASGTPGQRVTISCSGSSSNIGS |
| | SSYWMSWVRQAPGKGLEWVADIKQDGSEK | NTVNWSRQLPGTAPKLLIYSNNQRPSGVPDR |
| | YYVDSVKGRFTISGDNAKNSLYLQMNSLR | FSGSKSGTSASLAISGLQSEDEADYYCAAWD |
| | AEDTAVYYCAKDVGSTAINYVRAYTWFDP | DSINVYVFGTGTKVTVL |
| | WGQGTLVTVSS | (SEQ ID NO: 30) |
| | (SEQ ID NO: 9) | |
| YS4 | QVQLQESGGGLVQPGGSLRLSCAASGFTF | KIVLTQSPSSLSASVGDTVTIACRASRDIRN |
| | SNYAMSWVRQAPGKGLEWVSTISGSGSST | DLAWYQQKPGKAPKLLIYGASSLQSGVPSRF |
| | FYVDSVKGRFTISRDNSKNTLYLQMNSLR | SGSGSGTEFILTISSLQPEDFATYYCHRINS |
| | AEDTAVYYCAQGLYSSGWANWFDPRGQGT | YPLTFGGGTKVDIK |
| | LVTVSS | (SEQ ID NO: 31) |
| | (SEQ ID NO: 10) | |
| YS8 | QVQLQESGGGVVQPGRSLRLSCAASGFTF | NFMLTQPASLSGSPGQSITISCTGTSSDVGG |
| | SSYGMHWVRQAPGKGLEWVAVISYDGSNK | YNYVSWYQQHPGYAPKLMIYDVSNRPSGVSN |
| | YYADSVKGRFTISRDNSKNTLYLQMNSLR | RFSGSKSGNTASLTISGLQAEDEADYYCSSY |
| | AEDTAVYYCAKVMGLAAAGLDAFDIWGQG | TSSSTPWVFGGGTKLTVL |
| | TTVTVSS | (SEQ ID NO: 32) |
| | (SEQ ID NO: 11) | |
| YS7 | QVQLVQSGGGVVQPGRSLRLSCAASGFTF | SYVLTQDPAVSVALGQTVRITCQGDSLRSYY |
| | SSYAMHWVRQAPGKGLEWVAVISYDGSNK | ASWYQQKPGQAPVLVIYGKNNRPSGIPDRFS |
| | YYADSVKGRFTISRDTSTNTLYLQMNSLR | GSSSGNTASLTITGAQAEDEADYYCNSRDSS |
| | ADDTAVYYCGRESSGSPGVWGQGTTVTVS | GNQFGGGTKLTVL |
| | S | (SEQ ID NO: 33) |
| | (SEQ ID NO: 12) | |
| YS9 | QVQLVESGGGLIQPGGSLRLSCAASGFTV | SSELTQDPAVSVALGQTVRITCQGDSLRTYY |
| | SSNYMSWVRQAPGKGLEWVSVIYTDGSTY | ASWYQQRPGQAPILVLYGKNNRPSGIPDRFS |
| | YADSVKGRFTISRDNSKNTLYLQMNSLRA | GSSSGNTASLTITGAQAEDEADYYCNSRDSS |
| | EDTAIYYCARDRGTSGYDWAWFDLWGQGT | GNHVVFGGGTKLTVL |
| | LVTVSS | (SEQ ID NO: 34) |
| | (SEQ ID NO: 13) | |
| YS10 | QVQLQESGGGLVQPGGSLRLSCAASGFTF | QSVLTQPASVSGSPGQSITISCTGTGSDVGS |
| | SSYAMSWVRQAPGKGLEWVSAISGSGGST | YNYVSWYQQNPGKAPKLMIYEVSNRPSGVSN |
| | YYADSVKGRFTISRDNSKNTLYMQMNSLR | RFSGSKSGNTASLTISGLQAEDEADYYCSSY |
| | AEDTAVYYCAKDRYYYGSGKDAFDIWGRG | TTSSTLVFGGGTKVTVL |
| | TMVTVSS | (SEQ ID NO: 35) |
| | (SEQ ID NO: 14) | |
| YS11 | QVQLVESGGGLVQPGGSLGLSCAASGFTF | SELTQDPAVSVALGQTVRITCQGDSLRSYYA |
| | SNYWMSWVRQAPGKGLEWVANVRQDGGQK | SWYQQKPGQAPVLVIYGENSRPSGIPDRFSG |
| | YYVDSVKGRFTISRDNAKNSLYLQMNSLR | SSSGNTASLTITGAQAEDEADYYCNSWDSSG |
| | TEDTAVYFCVSQRNSGEHDYWGQGTLVTV | NHVVFGGGTKLTVL |
| | SS | (SEQ ID NO: 36) |
| | (SEQ ID NO: 15) | |
| 3G7HY | EVQLVESGGGLVQPGGSLRLSCAASGFTF | AIRMTQSPSSLSASVGDRVTITCRASQSISS |
| | SDYYMSWIRQAPGKGLEWVSYISSSGSTI | YINWYQQKPGKAPKLLIYAASSLQSGVPSRF |
| | YYADSVKGRFTISRDNSKNTLYLQMNSLR | SGSGSGTDFTLTISSLQPEDFATYYCQQSYS |
| | AEDTAVYYCARDYGRIAAAGRHYWGQGTL | TPRTFGQGTKLEIK |
| | VTVSS | (SEQ ID NO: 37) |
| | (SEQ ID NO: 16) | |
| 3G7NY | EVQLVESGGGLVQPGGSLRLSCAASGFTF | DIVMTQSPLSLPVTPGEPASISCRSSQSLLH |
| | SDYYMSWIRQAPGKGLEWVSYISSSGSTI | SNGYDYLDWYLQKPGQSPQLLIYLGSNRASG |
| | YYADSVKGRFTISRDNSKNTLYLQMNSLR | VPDRFSGSGSGTDFTLKISRVETEDVGIYYC |
| | AEDTAVYYCARDYGRIAAAGRNYWGQGTL | MQGLQTPSFGQGTKLEIK |
| | VTVSS | (SEQ ID NO: 38) |
| | (SEQ ID NO: 17) | |
| 3G7 | QVQLQESGGGVVRPGGSLRLSCAASGFTF | SSELTQDPAVSVALGQTVRITCQGDSLRSYY |
| | SDYYMSWIRQAPGKGLEWVSYISSSGSTI | ASWYQQKPGQAPVPVIYGKNNRPSGIPDRFS |
| | YYADSVKGRFTISRDNSKNTLYLQMNSLR | GSSSGNTASLTITGAQAEDEADYYCNSRDSS |
| | AEDTAVYYCARDYGRIAAAGRHYWGQGTL | STHRGVFGGGTKLTVL |
| | VTVSS | (SEQ ID NO: 39) |
| | (SEQ ID NO: 18) | |
| SB2 | EVQLVESGGGLVKPGGSLRLSCAASGFTF | DIQLTQSPSSLSASVGDRVTITCRASRSIST |
| | SDYYMSWIRQAPGKGLEWVSYISSSGSSI | YLSWYQQKPGKAPKLLIYDASRLQNGVPSRF |
| | YYADSVKGRFTISRDNAKNSLYLQMNSLK | SGSGSDTDFTLTISSLQPEDFATYFCQQSYN |
| | AEDTAVYYCARDITDVVGVSFDYWGQGTL | PPWTFGQGTKLEIK |
| | VTVSS | (SEQ ID NO: 40) |
| | (SEQ ID NO : 19) | |
| 2C8 | EVQLVESGGGVVQPGRSLRLSCAASGFTF | QSALTQPASVSGSPGQSITISCTGTSSDVGG |
| | SSYGMHWVRQAPGKGLEWVAVISYDGSNK | YNYVSWYQQHPGKAPKLMIYDVSNRPSGVSN |
| | YYADSVKGRFTISRDNSKNTLYLQMNSLR | RFSGSKSGNTASLTISGLQAEDEAYYYCSSY |
| | AEDTAEYYCAKVMGLAAAGLDAFDIWGQG | TSSSDPWVFGGGTQLTVL |
| | TLVTVSS | (SEQ ID NO: 41) |
| | (SEQ ID NO: 20) | |
| UA8kappa | EVQLVESGGGVVQPGRSLRLSCAASGFTF | NIQMTQSPSSLSASVGDRVTITCRAGQPIST |
| | SSFGMHWVRRAPGKGLEWVAVISYDGSNQ | YVNWYQHKPGKAPKLLIYGASNLQSGVPSRF |
| | YYADSVKGRFTISRDNSKNTLYLQMNSLR | SGGGSATDFTLTISSLQPEDFATYYCQQSYS |
| | AEDTAVYYCGSRPGGGYASGSTVAYWGQG | SLLTFGDGTKVEIK |
| | TLVTVSS | (SEQ ID NO: 42) |
| | (SEQ ID NO: 21) | |

In various embodiments the antibodies comprise the three VH CDRs and/or the three VL CDRs of antibodies 3051.1, G12FC3, M6c42b, 4F3YW, M40pr146, UA20, UA8, 585II41, 585II41.1, 585II56, 3076, 3051, M49R, RCI-14, II79_4, II79_3, T5II-4B.1, T5II-4B3.2, RCI-11, RCI-20, CI-11A, CI-14A, or S95-2 that are described in PCT/US2008/076704 (WO 2009/039192) or the mPA7 antibody. The amino acid sequences ofthe VH and VL chains of these antibodies and the CDRs comprising these domains are shown in PCT/US2008/076704 and the amino acid sequences of these domains are reproduced below in Table Error! Reference source not found.

The sequence shown below are scFv antibodies (the VL and VH regions are joined by a (Gly₄Ser)₃ (SEQ ID NO:43) linker, however it will be recognized that other antibody forms comprising the CDRs (or the VH and/or VL domains) are possible.

TABLE 2
Additional antibodies.

| | | SEQ ID |
|---|---|---|
| Clone | Amino Acid Sequence | No |
| 3051.1 | QVQLQESGGGLVKPGGPLRLSCAASGFTFSSYGMYWVRQAPGKGLEWV | 44 |
| | STLSRSGSGTYYADSVKGRFTISRDNSKNTLYLQMNSLRAEDTAVYYC | |
| | ASIAVAGNYFDYWGQGTLVTVSSGGGGSGGGGSGGGGSSYVLTQDPAV | |
| | SVALGQTVRITCQGDSLRSYYASWYQERPGQAPLLVIYGKNNRPSGIP | |
| | DRESGSNSGSTATLTISRVEAGDEGDYYCQVWDSINEQVVEGGGTKVT | |
| | VL | |
| G12FC3 | QVQLVQSGGGVVQPGRSLRLSCAATGIPFSGSGMHWVRQAPGKGLEWV | 45 |
| | TMIWYDGSNKFYADSVKGRFTISRDNSKNTLYLQMDSLRAEDTAVYFC | |
| | ARDKGVRSMDVWGLGTTVTVSSGGGGSGGGGSGGGGSNFMLTQPPSVS | |
| | VAPGQTAKITCDGYSIRTKSVHWYQQKPGQAPVVVVHDDSDRPSGIPE | |
| | RFSGSNSGTTATLTISRVEAGDEADYYCQAWDSISEEVVFGGGTKLTV | |
| | L | |
| M6c42b | QVQLQESGGGLVQPGGSLRLSCSASGFTFGTYAMRWVRQTSGKGLEWV | 46 |
| | SGIGVSGDAYYTDSVRGRFTISRDNSKNTLYLQMNTLRAEDTATYYCT | |
| | RKSSTTSNDYWGRGTLVTVSSGGGGSGGGGSGGGGSSYVLTQDPAVSV | |
| | ALGQTVRITCQGDNIGSKSVHWYQQKPGQAPVLVVYDDSDRPSGIPER | |
| | FSGSNSGTTATLTISSVEAGDEADYYCQAWDSISEHVIFGGGTKVTVL | |
| 4F3YW | QVQLQESGGGLVQPGGSLRLSCAASGFTFSSYAMHWVRQAPGKGLEWV | 47 |
| | AVISYDGSNKYYADSVKGRFTISRDNSKNTLYLQMNSERAEDTAVYYC | |
| | ARFSSGWYYFDYWGQGTLVTVSSGGGGSGGGGSGGGGSDIQMTQSPSF | |
| | LSASVGDRITITCRASHDISSYFAWYQQKPGKAPKPLIYAASTLQSGV | |
| | PSRFSGSGSGTEFTLTISSLQPEDFATYYCQQLGSYPLTFGGGTKLEI | |
| | K | |
| M40pr146 | QVQLLQSGGGLVQPGGSLRLSCAASGFTFSSYAMSWVRQAPGKGLEWV | 48 |
| | SAISGSGGSTYYTDSVKGRFTISRDNSKNTLYLQMNSLRAEDTAVYYC | |
| | AKSHDYGDYAGFDYWGQGTLVTVSSGGGGSGGGGSGGGGSHVILTQDP | |
| | AVSVALGQTVRITCQGDSLKSYYASWYQQKPGQAPVLVIYGKNNRPSG | |
| | IPDRFSGSSSGTTASLTITGAQAEDEADYYCHSRDSSGTHLRVFGGGT | |
| | KLTVL | |
| UA20 | QVQLQESGGGLVKPGGSLRLSCAASGFTFSNAWMNWVRQAPGKGLEWV | 49 |
| | GRIKSKTDEGTTDYAAPVKGRFSISRDDSKNTLYLQMNSLKTEDTGVY | |
| | YCTATKGLGGSKLGQGTLVTVSSGGGGSGGGGSGGGGSQSVLTQPPSA | |
| | SGTPGQRVTISCSGSSSNIGNNTVNWSRQLPGTAPKLLIYSNDQRPSG | |
| | VPDRFSGSKSGTSASLAITGLQPEDEADYYCGTWDSSLSAYVFGTGTK | |
| | LTVL | |
| UA8 | QVQLVESGGGVVQPGRSLRLSCAASGFTFSSFGMHWVRRAPGKGLEWV | 50 |
| | AVISYDGSNQYYADSVKGRFTISRDNSKNTLYLQMNSLRAEDTAVYYC | |
| | GSRPGGGYASGSTVAYWGQGTPVTVSSGGGGSGGGGSGGGGSSSELTQ | |
| | DPAVSVALGQTVRITCQGDSLRSYYASWYQQKPGQAPLLVIYGQNIRP | |
| | SGIPDRFSGSSSGNSASLTITGAQAEDEADYYCHSRDSSGKYVFGVGT | |
| | KVTVL | |
| 585II41 | QVQLVESGGGLVQPGGSLRLSCAASGFTFSSYAMGWVRQAPGKGLEWV | 51 |
| | SAISGSGGSTYYADSVKGRFTISRDNSKDTLYLQMNSLRAEDTAVYYC | |
| | ASRSLLDYWGQGTLVTVSSGGGGSGGGGSGGGGSNFMLTQDPAVSVAL | |
| | GQTVRITCQGDSLRSYYASWYQQKPGQAPLLVIYGKNNRPSGIPDRFS | |
| | GSSSGNTASLTITGAQAEDEADYYCNSRDSSGNPVFGGGTKVTVL | |
| 585II41.1 | QVQLVESGGGLVQPGGSLRLSCAASGFTFSSYAMSWVRQAPGKGLEWV | 52 |
| | SAISGSGGSTYYADSVKGRFTISRDNSKDTLYLQMNSLRAEDTAVYYC | |
| | ASRSLLDYWGQGTLVTVSSGGGGSGGGGSGGGGSNFMLTQDPAVSVAL | |
| | GQTVRITCQGDSLRSYYASWYQQKPGQAPLLVIYGKNNRPSGIPDRFS | |
| | GSSSGNTASLTITGAQAEDEADYYCNSRDSSGNPVFGGGTKVTVL | |
| 585II56 | QVQLQESGGGLVQLGGSLRLSCAASGFTFSSYAMSWVRQAPGKGLEWV | 53 |
| | SAISGSGGSTYYADSVKGRFTISRDNSKNTLYLQMSSLRAEDTAFYYC | |
| | ANSAYTGGWYDYWGHGTLVTVSSGGGGSGGGGSGGGGSSSELTQDPAV | |
| | SVALGQTVKITCQGDSLRTYYASWYQQRPGQAPVLVIYGENSRPSGIP | |
| | DRESGSSSGNTASLTITGAQAEDEADYYCNSRDSSGNHLRVFGGGTKL | |
| | TVL | |
| 3076 | QVNLRESGGGLVQPGGFLRLSCAAFGFTFSGYWMSWVHPAPGKGLEWV | 54 |
| | ANIKQDGSEKFYVDSVKGRFTISRDNAKNSLFLQMNSLRAEDTAVYFC | |
| | ARGLLSDYWGQGTLVPVSSGGGGSGGGGSGGGGSNFMLTQPPSVSVAP | |
| | GKTASLTCGGYNIGTKSVHWYQQKPGQAPVVVVHDDSDRPSGIPERFS | |
| | GSNSGTTATLTIIRVEAGDEADYYCQAWDSISEEVVFGGGTKLTVL | |
| 3051 | QVQLQESGGGLVKPGGPLRLSCAASGFTFSSYGMYWVRQAPGKGLEWV | 55 |
| | STLSRSGSGTYYAESVKGRFTISRDNSKNTLYFQMNSLRAEDTAVYYC | |
| | ASIAVAGNYFEYWGQGTLVTVSSGGGGSGGGGSGGGGSSYVLTQDPAV | |
| | SVALGQTVRITCQGDSLRSYYASWYQERPGQAPLLVIYGKNNRPSGIP | |
| | DRFSGSNSGSTATLTISRVEAGDEGDYYCQVWDSINEQVVFGGGTKVT | |
| | VL | |
| M49R | QVQLQESGGGLVKPGESLRLSCAASGFTFSDHYMDWVRQAPGKGLEWV | 56 |
| | AYIRYDGSTKYYADSVKGRFTISRDNSKNTLYLQMNSLRPEDTAFYYC | |
| | ARLIAEAEGWFDPWGQGTLVTVSSGGGGSGGGGSGGGGSNFMLTQPPS | |
| | VSVAPGKTARITCGGNNIGSKSVYWYQQKPGQAPVLVVYDDSDRPSGI | |
| | PERFSGSNSGNTATLTISRVEAGDEADYYCQVWDSSSDHVVFGGGTKV | |
| | TVL | |
| RCI-14 | QVQLLQSAGGLVQPGGSLRLSCAASGFTFSTYAMNWVRQAPGKGLEWV | 57 |
| | SGISGSGGSTNYADSVKGRFTISRDSSKNTLFLQMNSLRAEDTAVYYC | |
| | AKDYGSGWYDYWGQGTLVTVSSGGGGSGGGGSGGGGSSSELTQDPAVS | |
| | VALGQTVRITCQGDSLRSYYASWYQERPGQAPLLVIYGRNERPSGIPD | |
| | RFSASSSGNTASLTITGAQAEDEADYYCQVWDSFNEQVVFGGGTKLTV | |
| | L | |
| II79_4 | QVQLVESGGGLVQPGGSLRLSCAASGFTFSSYAMSWVHQAPGKGLEWV | 58 |
| | SAISGSGGSTYYADSVKGRFTISRDNSKNTLYLQMNSLRAEDTAVYYC | |
| | AKTYYGFWSGYYDYLGQGTLVTVSSGGGGSGGGGSGGGGSSSELTQDP | |
| | AVSVGLGQTVTITCQGDSLRSYYANWYQQKPGQAPILVIYGENNRPSG | |
| | IPDRFSGSSSGNTASLTITGAQAEDEADYYCHSRDSSGTHLRVEGGGT | |
| | KLTVL | |
| II79_3 | QVQLLESGGGVVQPGTSLRLSCAASGFTFSNYAINWVRQAAGKGLEWV | 59 |
| | SGISGSGVSTSYADSVKGRFTVSRDNSKNTLYLQMNSLRVEDTALYYC | |
| | AKNGGGPEYLQHWGQGTLVTVSSGGGGSGGGGSGGGGSQSVLTQPPSA | |
| | SGTPGQRVTISCSGSSSNIGNNTVNWSRQLPGTAPKLLIYSNDQRPSG | |
| | VPDRFSGSKSGTSASLAITGLQPEDEADYYCGTWDSSLSAYVEGTGTK | |
| | LTVL | |
| TSII-4B.1 | QVQLQESGGTLVQPGGSLRLSCAASGFTFSSYAMSWVRQAPGRGLEWV | 60 |
| | STISGSGGSTYYADSVKGRFTISRDNSKNTLYLQMNSLRAEDTAVYYC | |
| | AKGAYSGSYWGQGTLVTVSSGGGGSGGGGSGGGGSSSELTQDPAVSVA | |
| | LGQTVRITCQGDSLRSYYASWYQQKPGQAPSLVIYGENSRPSGIPDRF | |
| | SGSSSGNTASLTITGAQAENEADYYCQAWDSSTAVVFGGGTKLTVL | |
| TSII-4B.2 | QVQLQESGGTLVQPGGSLRLSCAASGFTFSSYAMSWVRQAPGRGLEWV | 61 |
| | STISGSGGSTYYADSVKGRFTISRDNSKNTLYLQMNSLRAEDTAVYYC | |
| | AKGAYSGSHWGQGTLVTVSSGGGGSGGGGSGGGGSSSELTQDPAVSVA | |
| | LGQTVRITCQGDSERSYYASWYQQKPGQAPSLVIYGENSRPSGIPDRF | |
| | SGSSSGNTASLTITGAQAENEADYYCQAWDSSTAVVFGGGTKLTVL | |
| RCI-11 | QVQLVESGAEVKKPGASVKVSCKASGYTFTSYGISWVRQAPGQGLEWM | 62 |
| | GWISAYNGNTNYAQKLQGRVTMTTDTSTSTAYMELRSLRSDDTAVYYC | |
| | ARPIYDSSGYDAFDIWGQGTMVTVSSGGGGSGGGGSGGGGSDIVMTQS | |
| | PSTLSASIGDRVTITCRASEGIYHWLAWYQQKPGKAPKLLIYKASSLA | |
| | SGAPSRFSGSGSGTDFTLTISSLQPDDFATYYCQQYHTISRTFGPGTK | |
| | VDIK | |
| RCI-20 | QVQLVESGGGLVKPGGSLRLSCAASGFTFSSYAMHWVRQAPGKGLEWV | 63 |
| | AVISYDGSNKYYADSVKGRFTISRDNSKNTLYLQMNSLRAEDTAVYFC | |
| | VRPSDSGWSFEHWGQGTLVPVSSGGGGSGGGGSGGGGSQSVLTQPPSA | |
| | SGTPGQRVTISCSGSSSNIGNNTVNWSRQLPGTAPKLLIYSNDQRPSG | |
| | VPDRFSGSKSGTSASLAITGLQPEDEADYYCGTWDSSLSAYVEGTGTK | |
| | LTVL | |
| CI-11º | QVQLQESGGGLVQPGGSLRLSCAASGFTFSSYAMSWVRQAPGKGLEWV | 64 |
| | AVISYDGSNKYYADSVKGRFTISRDNSKNTLYLQMNSLRAEDTAVYYC | |
| | VRGDRSYGAEYFQHWGQGTLVTVSSGGGGSGGGGSGGGGSSSELTQDP | |
| | AVSVASGQTVRITCQGDSLRSYYASWYQQKPGQAPLLVIYGKNIRPSG | |
| | IPDRFSGSTSGNSASLTITGAQAEDEADYYCNSRDSSGNRNWVFGGGT | |
| | KLTVL | |
| CI-14° | QVQLQESGGGLVKPGGSLRLSCAASGFTSSSYAMHWVRQAPGKGLEYV | 65 |
| | SAIGGNGGTYYADSVKGRFTISRDNSKNTLYLQMNSLRAEDTAVYYCA | |
| | KEGEQWLEYRYYYGMDVWGQGTTVTVSSGGGGSGGGGSGGGGSSSELT | |
| | QDPAVSVALGQTVRITCQGDSLRSYYASWYQQKPGQAPSLVIYGENSR | |
| | PSGIPDRESGSSSGNTASLTITGAQAENEADYYCQAWDSSTAVVFGGG | |
| | TKLTVL | |
| $95-2 | QVQLVESGGGVVQPGRSLRLSCTASGFTFSSYGMHWVRQAPGKGLEWV | 66 |
| | AVISYDGSNKYYADSVKGRFTISRDNSKNTLYLQMNSLRAEDTAVYYC | |
| | ARGGRYSSNWFSYYYYGMDVWGQGTTVTVSSGGGGSGGGGSGGGGSNF | |
| | MLTQPPSVSVAPGKTARITCGGNNIGSKSVYWYQQKPGQAPVLVVYDD | |
| | SDRPSGIPERFSGSNSGNTATLTISRVEAGDEADYYCQVWDSSSDHVV | |
| | FGGGTKVTVL | |

Using the amino acid sequences provided for the YS5, YS5F, YS5v1D, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and UA8kappa antibodies, numerous antibody forms can be prepared, e.g., as described below. Such forms include, but are not limited to a substantially intact (e.g., full length) immunoglobulin (e.g., an IgA, IgE, IgG, and the like), an antibody fragment (e.g., Fv, Fab, (Fab′)₂, (Fab′)₃, IgGΔCH₂, a minibody, and the like), a single chain antibody (e.g., scFv), a diabody, a unibody, an affibody, and the like.

It will be recognized, that where the antibodies are single chain antibodies, the VH and VL domains comprising such antibody can be joined directly together or by a peptide linker. Illustrative peptide linkers include, but are not limited to GGGGS GGGGS GGGGS (SEQ ID NO:67), GGGGS GGGGS (SEQ ID NO:68), GGGGS (SEQ ID NO:69), GS GGGGS GGGGS GGS GGGGS (SEQ ID NO:70), SGGGGS (SEQ ID NO:71), GGGS (SEQ ID NO:72), VPGV (SEQ ID NO:73), VPGVG (SEQ ID NO:74), GVPGVG (SEQ ID NO:75), GVG VP GVG (SEQ ID NO:76), VP GVG VP GVG (SEQ ID NO:77), GGSSRSS (SEQ ID NO:78), and GGSSRSSSSGGGGSGGGG (SEQ ID NO:79), and the like.

As indicated above, in various embodiments, the antibody binds (e.g., specifically binds CD46 (e.g., domains 1 and/or 2). Typically antibodies contemplated herein will specifically bind prostate cancer cells including, but not limited to cells of a cell line selected from the group consisting of DU145 cells, PC3 cells, and LnCaP cells. In certain embodiments the antibody binds to a prostate tumor cell with an affinity greater than (K_{D} less than) about 5 nM when measured on live prostate tumor cells by FACS. In certain embodiments the affinity is greater than (K_{D} less than) about 1 nM, or at about 100 pM, or about 50 pM, or about 10 pM, or about 1 pM.

Using the sequence information provided herein antibodies comprising one or more of the CDRs comprising, e.g., YS5, YS5F, YS5v1D, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and UA8kappa, or antibodies comprising the VH and/or VL domain(s) of these antibodies can readily be prepared using standard methods (e.g. chemical synthesis methods and/or recombinant expression methods) well known to those of skill in the art, e.g., as described below.

In addition, other “related” prostate cancer specific antibodies can be identified by screening for antibodies that bind to the same epitope (e.g. that compete with one or more of YS5, YS5F, YS5v1D, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and/or UA8kappa antibodies for binding to CD446 and/or to a cell expressing or overexpressing CD46, e.g., a prostate cancer cell) and/or by modification of the YS5, YS5F, YS5v1D, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and/or UA8kappa antibodies identified herein to produce libraries of modified antibody and then rescreening antibodies in the library for improved binding to and/or internalization into cells expressing or overexpressing CD46, e.g., prostate cancer cells.

In some embodiments, that antibody is a recombinant antibody (or antigen binding fragment thereof) that specifically binds CD46. In some embodiments, antibody or antigen binding fragment or variant thereof is a monoclonal antibody. In some embodiments, antibody or antigen binding fragment or variant thereof is a human antibody, a murine antibody, a humanized antibody, or a chimeric antibody. In some embodiments, the antibody comprises or consists of a function fragment of a full length antibody (e.g., an antigen binding fragment of a full length antibody) such as a monovalent Fab, a bivalent Fab′2, a single-chain variable fragment (scFv), or functional fragment or variant thereof. In some embodiments, the recombinant antibody (or antigen binding fragment thereof) comprises an immunoglobulin variable heavy chain domain (VH). In some embodiments, the recombinant antibody (or antigen binding fragment thereof) comprises an immunoglobulin variable light chain domain (VL). In some embodiments, the recombinant antibody (or antigen binding fragment thereof) comprises a VH and a VL.

In some embodiments, the antibody (or antigen binding fragment thereof) comprises an Fc region. In some embodiments, the antibody (or antigen binding fragment thereof) is a full length antibody. In some embodiments, the antibody (or antigen binding fragment thereof) comprises a first light chain that comprises a light chain variable region and a light chain constant region; a first heavy chain that comprises a heavy chain variable region and a heavy chain constant region; a second light chain that comprises a light chain variable region and a light chain constant region; and a second heavy chain that comprises a heavy chain variable region and a heavy chain constant region. In some embodiments, the first and second light chains have at least 95%, 96%, 97%, 98%, 99%, or 100% sequence identity. In some embodiments, the first and second light chains bind the same epitope. In some embodiments, the first and second heavy chains have at least 95%, 96%, 97%, 98%, 99%, or 100% sequence identity. In some embodiments, the first and second heavy chains bind the same epitope.

In some embodiments, the antibody (or antigen binding fragment thereof) is derived from non-human (e.g. rabbit or mouse) antibodies. In some instances, the humanized form of the non-human antibody contains a minimal non-human sequence to maintain original antigenic specificity. In some cases, the humanized antibodies are human immunoglobulins (acceptor antibody), wherein the CDRs of the acceptor antibody are replaced by residues of the CDRs of a non-human immunoglobulin (donor antibody), such as rat, rabbit, or mouse donor having the desired specificity, affinity, avidity, binding kinetics, and/or capacity. In some instances, one or more framework region (FR) residues of the human immunoglobulin are replaced by corresponding non-human residues of the donor antibody.

In some embodiments, the CD46 binding antibody comprises an immunoglobulin variable heavy chain domain (VH) that comprises at least one, two, or three complementarity determining regions (CDRs) disclosed in Table 1, 2, or 3 or a sequence substantially identical thereto (e.g., a sequence that has at least 90%, 95%, 96%, 97%, 98%, or 99% sequence identity).

In some embodiments, the CD46 binding antibody comprises an immunoglobulin variable light chain domain (VL) that comprises at least one, two, or three complementarity determining regions (CDRs) disclosed in Table 1, 2 or 4 a sequence substantially identical thereto (e.g., a sequence that has at least 90%, 95%, 96%, 97%, 98%, or 99% sequence identity).

In some embodiments, the CD46 binding antibody comprises a VH that comprises at least one, two, or three complementarity determining regions (CDRs) disclosed in Table 1, 2, or 3 or a sequence substantially identical thereto (e.g., a sequence that has at least 90%, 95%, 96%, 97%, 98%, or 99% sequence identity); and a VL that comprises at least one, two, or three complementarity determining regions (CDRs) disclosed in Table 1, 2, or 4 or a sequence substantially identical thereto (e.g., a sequence that has at least 90%, 95%, 96%, 97%, 98%, or 99% sequence identity). For example, the CD46 binding antibody can comprise a VH that comprises at least one, two, or three complementarity determining regions (CDRs) disclosed in Table 3 or a sequence substantially identical thereto (e.g., a sequence that has at least 90%, 95%, 96%, 97%, 98%, or 99% sequence identity); and a VL that comprises at least one, two, or three complementarity determining regions (CDRs) disclosed in Table 4 or a sequence substantially identical thereto (e.g., a sequence that has at least 90%, 95%, 96%, 97%, 98%, or 99% sequence identity).

In some embodiments, the CD46 binding antibody comprises a VH that comprises a CDR1 of SEQ ID NO: 80, a CDR2 of SEQ ID NO: 81, and a CDR3 of SEQ ID NO: 82.

In some embodiments, the CD46 binding antibody comprises a VL that comprises a CDR1 of SEQ ID NO: 83, a CDR2 of SEQ ID NO: 84, and a CDR3 of SEQ ID NO: 85.

In some embodiments, the CD46 binding antibody comprises a VH that comprises a CDR1 of SEQ ID NO: 80, a CDR2 of SEQ ID NO: 81, and a CDR3 of SEQ ID NO: 82; and a VL that comprises a CDR1 of SEQ ID NO: 83, a CDR2 of SEQ ID NO: 84, and a CDR3 of SEQ ID NO: 85.

TABLE 3
VH CDR amino acid sequences of anti-CD46
antibodies as defined by Kabat et al.

| | SEQ | | SEQ | | SEQ | |
|---|---|---|---|---|---|---|
| | ID | | ID | | ID | |
| Antibody | NO | CDR1 | NO | CDR2 | NO | CDR3 |
| YS5FL | 80 | GLTVNNYA | 81 | ISYD | 82 | AKGG |
| | | | | GNNK | | GYFDL |

Note
“YS5FL” and “YS5” refer to the same antibody, which differs at some positions from “YS5F”.

TABLE 4
VL CDR amino acid sequences of anti-CD46
antibodies as defined by Kabat et al.

| | SEQ | | SEQ | | SEQ | |
|---|---|---|---|---|---|---|
| | ID | | ID | | ID | |
| Antibody | NO | CDR1 | NO | CDR2 | NO | CDR3 |
| YSSFL | 83 | SSNIGAGYD | 84 | GNN | 85 | SSYTSGTWL |

YS5FL has been found to bind specifically to the surface of LnCap-C4-2B, LnCap-C4, DU145, PC3-luc, and Hs27 prostate cancer cells, but not to non-tumor BPH1 cells. Likewise, YS5FL binds specifically to the surface of RPMI8226, MM.1S, MM.1R, and INA6 multiple myeloma cells.

In some embodiments, a CDR described herein comprises one, two, or three amino acid modifications. In some embodiments, said modification is a substitution, addition, or deletion. In some embodiments, a CDR described herein comprises one, two, or three conservative amino acid substitutions. In some embodiments, the one, two, or three amino acid modifications does not substantially modify binding to human CD46. In some embodiments, the one, two, or three amino acid modifications modifies binding to human CD46. In some embodiments, a VH-CDR3 and/or VL CDR3 comprises an amino acid substitution that modifies binding to human CD46, immunogenicity, or some other feature. In some embodiments, the amino acid substitution is an alanine (A).

In some embodiments, the CD46 binding antibody comprises a VH that comprises an amino acid sequence disclosed in Table 5 or a sequence substantially identical thereto (e.g., a sequence that has at least 90%, 95%, 96%, 97%, 98%, or 99% sequence identity).

In some embodiments, the CD46 binding antibody comprises a VL that comprises an amino acid sequence disclosed in Table 6 or a sequence substantially identical thereto (e.g., a sequence that has at least 90%, 95%, 96%, 97%, 98%, or 99% sequence identity).

In some embodiments, the CD46 binding antibody comprises a VH that comprises an amino acid sequence disclosed in Table 5 or a sequence substantially identical thereto (e.g., a sequence that has at least 90%, 95%, 96%, 97%, 98%, or 99% sequence identity); and a VL that comprises an amino acid sequence disclosed in Table 6 or a sequence substantially identical thereto (e.g., a sequence that has at least 90%, 95%, 96%, 97%, 98%, or 99% sequence identity).

In some embodiments, the CD46 binding antibody comprises a VH that comprises an amino acid sequence of SEQ ID NO: 86, or a sequence substantially identical thereto (e.g., a sequence that has at least 90%, 95%, 96%, 97%, 98%, or 99% sequence identity).

In some embodiments, the CD46 binding antibody comprises a VL that comprises an amino acid sequence of SEQ ID NO: 87, or a sequence substantially identical thereto (e.g., a sequence that has at least 90%, 95%, 96%, 97%, 98%, or 99% sequence identity).

In some embodiments, the CD46 binding antibody comprises a VH that comprises an amino acid sequence of SEQ ID NO: 86, or a sequence substantially identical thereto (e.g., a sequence that has at least 90%, 95%, 96%, 97%, 98%, or 99% sequence identity); and a VL that comprises an amino acid sequence of SEQ ID NO: 87, or a sequence substantially identical thereto (e.g., a sequence that has at least 90%, 95%, 96%, 97%, 98%, or 99% sequence identity).

TABLE 5
Amino acid sequence of the anti-CD46
variable heavy chain binding domains.

| | SEQ | |
|---|---|---|
| Name | ID NO | Amino Acid Sequence |
| YS5FL | 86 | QVQLVQSGGGVVQPGRSLRLACAASGLTVNNYAMHW |
| | | VRQAPGKGLEWVAVISYDGNNKYYADSVKGRFTISR |
| | | DNSKNTLYLQMNSLRAEDTAVYYCAKGGGYFDLWGR |
| | | GTLVTVSS |

TABLE 6
Amino acid sequence of the anti-CD46
variable light chain binding domains.

| | SEQ | |
|---|---|---|
| | ID | |
| Name | NO | Amino Acid Sequence |
| YS5FL | 87 | QSVLTQPPSVSGAPGQRVTISCTGSSSNIGAGYDVH |
| | | WYQQLPGTAPKLLIYGNNNRPSGVPDRESGSKSGTS |
| | | ASLAITGLQAEDEADYYCSSYTSGTWLFGGGTKLTVL |

In some embodiments, the CD46 binding antibody comprises a heavy chain that comprises an amino acid sequence disclosed in Table 7 or a sequence substantially identical thereto (e.g., a sequence that has at least 90%, 95%, 96%, 97%, 98%, or 99% sequence identity).

In some embodiments, the CD46 binding antibody comprises a light chain that comprises an amino acid sequence disclosed in Table 8 or a sequence substantially identical thereto (e.g., a sequence that has at least 90%, 95%, 96%, 97%, 98%, or 99% sequence identity).

In some embodiments, the CD46 binding antibody comprises a heavy chain that comprises an amino acid sequence disclosed in Table 7 or a sequence substantially identical thereto (e.g., a sequence that has at least 90%, 95%, 96%, 97%, 98%, or 99% sequence identity); and a light chain that comprises an amino acid sequence disclosed in Table 8 or a sequence substantially identical thereto (e.g., a sequence that has at least 90%, 95%, 96%, 97%, 98%, or 99% sequence identity).

In some embodiments, CD46 binding antibody comprises a heavy chain that comprises an amino acid sequence of SEQ ID NO: 88, or a sequence substantially identical thereto (e.g., a sequence that has at least 90%, 95%, 96%, 97%, 98%, or 99% sequence identity).

In some embodiments, the CD46 binding antibody comprises a light chain that comprises an amino acid sequence of SEQ ID NO: 89, or a sequence substantially identical thereto (e.g., a sequence that has at least 90%, 95%, 96%, 97%, 98%, or 99% sequence identity).

In some embodiments, the CD46 binding antibody comprises a heavy chain that comprises an amino acid sequence of SEQ ID NO: 88, or a sequence substantially identical thereto (e.g., a sequence that has at least 90%, 95%, 96%, 97%, 98%, or 99% sequence identity); and a light chain that comprises an amino acid sequence of SEQ ID NO: 89, or a sequence substantially identical thereto (e.g., a sequence that has at least 90%, 95%, 96%, 97%, 98%, or 99% sequence identity).

TABLE 7
Amino acid sequence of the anti-CD46 heavy chain.

| | SEQ | |
|---|---|---|
| | ID | |
| Name | NO | Amino Acid Sequence |
| YS5FL | 88 | QVQLVQSGGGVVQPGRSLRLACAASGLIVNNYAM |
| | | HWVRQAPGKGLEWVAVISYDGNNKYYADSVKGRF |
| | | TISRDNSKNTLYLQMNSLRAEDTAVYYCAKGGGY |
| | | FDLWGRGTIVTVSSASTKGPSVFPLAPSSKSTSG |
| | | GTAALGCLVKDYFPEPVTVSWNSGALTSGVHTEP |
| | | AVLQSSGLYSLSSVVTVPSSSLGTQTYICNVNHK |
| | | PSNTKVDKKVEPKSCDKTHTCPPCPAPELLGGPS |
| | | VFLFPPKPKDTLMISRTPEVTCVVVDVSHEDPEV |
| | | KENWYVDGVEVHNAKTKPREEQYNSTYRVVSVLT |
| | | VLHQDWINGKEYKCKVSNKALPAPIEKTISKAKG |
| | | QPREPQVYTLPPSREEMTKNQVSLTCLVKGFYPS |
| | | DIAVEWESNGQPENNYKTTPPVLDSDGSFFLYSK |
| | | LTVDKSRWQQGNVESCSVMHEALHNHYTQKSLSL |
| | | SPGK |

TABLE 8
Amino acid sequence of the anti-CD46 light chain.

| | SEQ | |
|---|---|---|
| | ID | |
| Name | NO | Amino Acid Sequence |
| YS5FL | 89 | QSVLTQPPSVSGAPGQRVTISCTGSSSNIGAGYD |
| | | VHWYQQLPGTAPKLLIYGNNNRPSGVPDRESGSK |
| | | SGTSASLAITGLQAEDEADYYCSSYTSGTWLFGG |
| | | GTKLTVLGQPKAAPSVTLEPPSSEELQANKATLV |
| | | CLISDFYPGAVTVAWKADSSPVKAGVETTTPSKQ |
| | | SNNKYAASSYLSLTPEQWKSHRSYSCQVTHEGST |
| | | VEKTVAPTECS |

In some embodiments, the anti-CD46 antibody disclosed herein comprises an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1 or IgG4. In some embodiments, the immunoglobulin constant region (e.g., the Fc region) is altered, e.g., mutated, to increase or decrease one or more of: Fc receptor binding, antibody glycosylation, the number of cysteine residues, effector cell function, or complement function.

### C. Chelators

The radioimmunoconjugates of the present invention include a chelator that chelates the radionuclide and that has a moiety that is or can be coupled to an antibody. Chelators for radionuclides are known to those of skill in the art. The chelator is typically a bifunctional chelator. As used herein, the term “bifunctional chelator” refers to a chelator that has a metal binding function as well as a chemically reactive functional group that provides the requisite chemistry for coupling to the antibody through a PEG linker.

In one embodiment, the chelator can be Macropa.NH2, which was developed by Thiele et al., (Thiele N A et al. (2017) Angew Chem Int Ed Engl, 56(46), 14712-14717), the teachings of which are incorporated herein by reference. The chelator can also be DOTA (1,4,7,10-Tetraazacyclododecane-1,4,7,10-tetraacetic acid; tetraxetan), and derivatives thereof such as p-SCN-Bn-DOTA and MeoDOTA-NCS or DOTP (1,4,7,10-Tetraazacyclododecane-1,4,7,10-tetra(methylene phosphonic) acid). In another embodiment, the chelator can be DFO or Desferoxamine (N′-[5-(acetyl-hydroxy-amino)pentyl]-N-[5-[3-(5-aminopentyl-hydroxy-carbamoyl)propanoylamino]-pentyl]-N-hydroxy-butane diamide). The chelator can also be NOTA (2,2′,2″-(1,4,7-triazacyclononane-1,4,7-triyl)triacetic acid).

In other embodiments, the chelator can include, but is not limited to, the following: isothiocyanatobenzyl-3-methyldiethylene triaminepentaacetic acid (NCS-DTPA) (see, e.g., PCT Publication No. WO94/11026), isothiocyanatobenzyl-1,4, 7, 10-Tetraazacyclododecane-1,4,7,10-tetra(methylenephosphonic acid) (p-NCS-DOTP) and Macropa-NCS (6-((16-((6-carboxypyridin-2-yl)methyl)-1,4, 10, 13-tetraoxa-7, 16-diazacyclooctadecan-7-yl)methyl)-4-isothiocyanatopicolinicacid).

Other examples of chelators that can be used include, but are not limited to, the following: 1,4, 7,10-Tetraazacyclododecane-1,4, 7-tris(aceticacid)-10-(2-thioethyl)acetamide (D03A), [(R)-2-Amino-3-(4-isothiocyanatophenyl)propyl]-trans-(S, S)-cyclohexane-1,2-diamine-pentaacetic acid (CHX-DTPA), 2-S-(4-Isothiocyanatobenzyl)-1,4, 7-triazacyclononane-1,4, 7-triacetic acid (SCN-NOTA), 1,4, 7-Triazacyclononane-1,4-bis-acetic acid-7-maleimidoethylacetamide (maleimide-NOTA), 4,11-bis(carboxymethyl)-1,4,8, 11-tetraazabicyclo[6.6.2]hexadecane)(CB-TE2A) and Triethylenetetramine (TETA) derivatives.

The chelator can be directly or indirectly coupled to the antibody. For example, the chelator can be coupled to the antibody by any chemical reaction that will bind the chelator and the antibody, so long as these retain their respective activities/characteristics for the intended use thereof. This coupling can include chemical mechanisms including for instance covalent binding, affinity binding, intercalation, coordinate binding and complexation. In one embodiment, the chelator is attached to the antibody through a PEG linker.

In one embodiment, each chelator carries one radionuclide. Optionally, each antibody is coupled to 1-3 chelator for an antibody:radionuclide ratio of 1:1 to 1:3. The number of chelators per antibody may be controlled for example by adjusting the pH of the reaction, the reaction time and the of fold excess of the chelator to antibody.

### D. Radionuclides

The complexes of the present invention include a radionuclide. The radionuclide is optionally an alpha emitter (a radionuclide that emits alpha particles), a beta emitter (a radionuclide that emits beta particles), or a gamma emitter (a radionuclide that emits gamma particles). Examples of radionuclides include, but are not limited to, ²²⁵Ac, ⁶⁷Cu, ¹⁷⁷Lu, ²¹³Bi, ⁹⁰Y, ¹⁸⁸Re, ⁴⁷Sc, ²²⁷Th, ²¹²Ph, ¹¹¹In, ¹²⁴I, ¹³¹I, ²¹³Bi, ⁸⁹Zr, ²¹¹At, ²¹²B, and ¹⁸⁶Re. Other suitable radionuclide suitable for use in the immunoconjugates disclosed herein will be known to those skilled in the art.

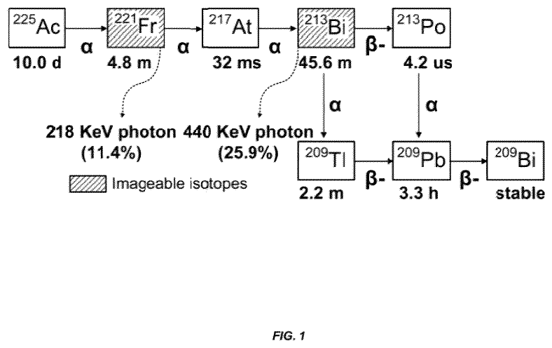

In one embodiment, the radionuclide is an alpha emitter (a radionuclide that emits alpha particles). The alpha-emitting radionuclide can include, but is not limited to, the following: ²²⁵Ac ²¹³Bi, ²²⁴Ra, ²¹²Pb, ²²⁷Th, ²²³Ra, ²¹¹At, and ¹⁴⁹T. In one embodiment, the radionuclide is Actinium-225 (²²⁵Ac), an alpha particle emitter. Use of ²²⁵Ac in the compositions of the present invention is particularly advantageous because it has a long half-life of 10 days (see, FIG. 1) due to its unique properties such as “nanogenerator” status and due to its unique ability to produce a total of 4α and 2β particles in its decay chain.

### E. Methods of Treating CD46 Expressing Cancers

In another aspect, provided herein are methods of treating certain cancers by administering to a subject an immunoconjugates described herein. As is known to those skilled in the art, expression of CD46 is low in normal cells, but is upregulated in human cancer cells such as ovarian cancer, breast cancer, lymphoma, hepatocellular carcinoma, lung cancer, prostate cancer, and colon cancer. The immunoconjugates described herein can be used to treat CD46 expressing cancers.

As used herein, “treating a cancer” includes, but is not limited to, reversing, alleviating or inhibiting the progression of the cancer or symptoms or conditions associated with the cancer. “Treating the cancer” also includes extending survival in a subject. Survival is optionally extended by at least 1, 2, 3, 6 or 12 months, or at least 2, 3, 4, 5 or 10 years over the survival that would be expected without treatment with a radioimmunoconjugate as described herein. “Treating the cancer” also includes reducing tumor mass and/or reducing tumor. Optionally, tumor mass and/or tumor burden is reduced by at least 5, 10, 25, 50, 75 or 100% following treatment with a radioimmunoconjugate as described herein. “Treating the cancer” also includes reducing the aggressiveness, grade and/or invasiveness of a tumor.

In one embodiment, the cancer is a CD46 expressing cancer. In some embodiments, the cancer is prostate cancer. In some embodiments, the cancer is castration resistant prostate cancer. In some embodiments, the cancer is metastatic prostate cancer.

In some embodiments, the cancer is multiple myeloma. In some embodiments, the cancer is relapsing multiple myeloma. In some embodiments, the cancer is remitting multiple myeloma. In some embodiments, the cancer is relapsing or remitting multiple myeloma.

In some embodiments, the cancer is lymphoma (including but not limited to Hodgkin's lymphoma), acute myeloid leukemia (AML), or metastatic renal cell carcinoma (mRCC).

As used herein, the terms “subject,” patient,” and “animal” include all members of the animal kingdom. In one embodiment, the subject is a mammal. In a further embodiment, the subject is a human being. In one embodiment, the subject is a patient having a disease, such as a cancer, e.g., a CD46 expressing cancer, such as prostate cancer.

In some embodiments, the immunoconjugates disclosed herein are administered for a period necessary to prevent occurrence or recurrence of disease, to alleviate symptoms, to diminish any direct or indirect pathological consequences of the disease, to prevent metastasis, to decrease the rate of disease progression, to ameliorate or palliate the disease state, and/or to bring about remission or to improve prognosis. In some embodiments, the period of time is (e.g., once or more a day for) 1-90 days, e.g., 1-60, 15-45, 5-15 days, e.g., 5-10 days, e.g., 3-10 days, e.g., 1, 2, 3, 4, 5, 6, 7, 8, 9, 10, 20, 30, 40, or 50 days.

Pharmaceutical compositions of the immunoconjugates as described herein can be prepared in accordance with methods well known and routinely practiced in the art. Pharmaceutically acceptable carriers are determined in part by the particular composition being administered, as well as by the particular method used to administer the composition. Accordingly, there is a wide variety of suitable formulations of pharmaceutical compositions described herein. Applicable methods for formulating the antibodies and determining appropriate dosing and scheduling can be found, for example, in Remington: The Science and Practice of Pharmacy, 21^{st} Ed., University of the Sciences in Philadelphia, Eds., Lippincott Williams & Wilkins (2005); and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, and Sustained and Controlled Release Drug Delivery Systems, J. R. Robinson, ed., Marcel Dekker, Inc., New York, 1978, each of which are hereby incorporated herein by reference. Pharmaceutical compositions are preferably manufactured under GMP conditions. Typically, a therapeutically effective dose or efficacious dose of the immunoconjugates (antibody and radionuclide) descried herein is employed in the pharmaceutical compositions. The immunoconjugates can be formulated into pharmaceutically acceptable dosage forms by conventional methods known to those of skill in the art. Dosage regimens are adjusted to provide the desired response (e.g., a therapeutic response). In determining a therapeutically or prophylactically effective dose, a low dose can be administered and then incrementally increased until a desired response is achieved with minimal or no undesired side effects. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A therapeutically effective amount of the antibodies and radionuclide will vary depending upon the subject and disease condition being treated, the weight and age of the subject, the severity of the disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, e.g., 1 to 10 mg/kg, 1.8 to 2.7 mg/kg of an anti-CD46 antibody described herein and/or antigen binding portion thereof, and/or immunoconjugate thereof as described herein. Dosage regiments may be adjusted to provide the optimum therapeutic response. An effective amount is also one in which any toxic or detrimental effects (i.e., side effects) of an antibody or antigen binding portion thereof or of the radionuclide are minimized and/or outweighed by the beneficial effects.

Pharmaceutical compositions as described herein can be administered by a variety of methods known in the art. The route and/or mode of administration vary depending upon the desired results. It is preferred that administration be intravenous, intramuscular, intraperitoneal, or subcutaneous, or administered proximal to the site of the target. The pharmaceutically acceptable carrier should be suitable for intravenous, intramuscular, subcutaneous, parenteral, intranasal, inhalational, spinal or epidermal administration (e.g., by injection or infusion). Depending on the route of administration, the active compound, e.g., antibody, may be coated in a material to protect the compound from the action of acids and other natural conditions that may inactivate the compound.

### F. Methods of Synthesizing the Radioimmunoconjugates

In another aspect, provided herein are methods for making the radioimmunoconjugates as disclosed herein. The methods generally involve coupling an antibody, such as an antibody that binds CD46, to a chelator through a PEGylated linker to produce an antibody-chelator conjugate (ACC), and then radiolabeling the ACC with a radionuclide.

As will be appreciated by those skilled in the art, the chelator can be coupled to the antibody (or a fragment or portion thereof) either through a lysine residue or a cysteine residue on the antibody and will depend, in part, on the functional group(s) present on end of the linker that will be attached to the antibody. Exemplary Chelator, Linker, Functional Groups, and Conjugation Reactions on YS5 are set forth in Table 9, infra.

TABLE 1
Exemplary Radioimmunoconjugate Components

| | | | Conjugation |
|---|---|---|---|
| | | | reaction |
| Chelator | Linker | Functional group | on YS5 |
| 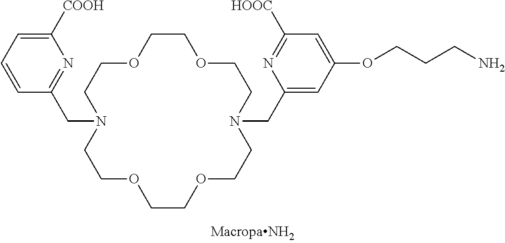 [SMILES: NCCCOc1cc(CN2CCOCCOCCN(Cc3cccc(C(=O)O)n3)CCOCCOCC2)nc(C(=O)O)c1] | PEG4, PEG6, PEG8 & PEG12 | 1. TFP ester 2. NHS ester 3. Maleimide | 1. Lysine 2. Lysine 3. Cysteine |
| 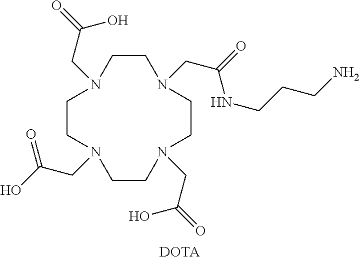 | PEG4, PEG6, PEG8 & PEG12 | 1. TFP ester 2. NHS ester 3. Maleimide | 1. Lysine 2. Lysine 3. Cysteine |
| 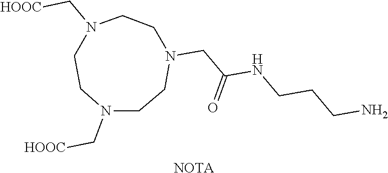 [SMILES: NCCCNC(=O)CN1CCN(CC(=O)O)CCN(CC(=O)O)CC1 ; "NOTA"] | PEG4, PEG6, PEG8 & PEG12 | 1. TFP ester 2. NHS ester 3. Maleimide | 1. Lysine 2. Lysine 3. Cysteine |
| 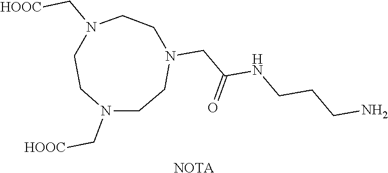 [SMILES: NCCCNC(=O)CN1CCN(CC(=O)O)CCN(CC(=O)O)CC1 ; "NOTA"] | PEG4, PEG6, PEG8 & PEG12 | 1. TFP ester 2. NHS ester 3. Maleimide | 1. Lysine 2. Lysine 3. Cysteine |

When the chelator is to be coupled or attached to a lysine residue on the antibody, the following exemplary reaction scheme can be used:

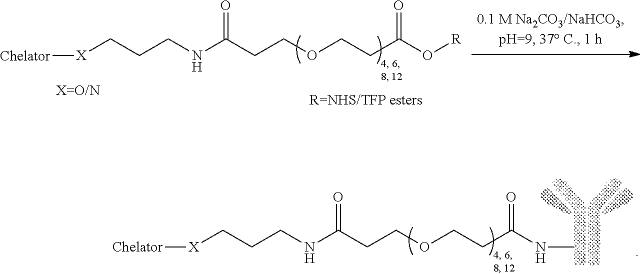

When the chelator is to be coupled or attached to a cysteine residue on the antibody, the following exemplary reaction scheme can be used:

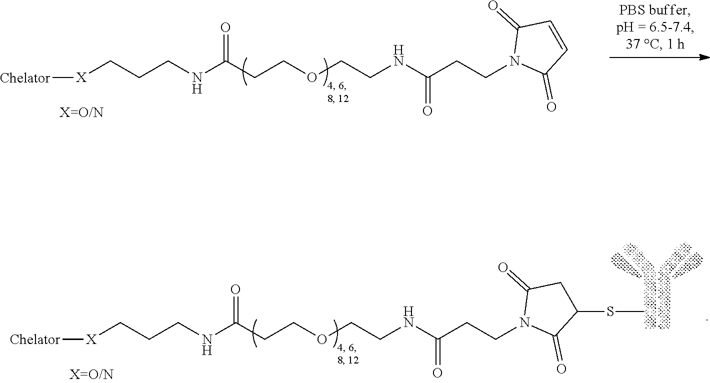

When the chelator is DOTA or NOTA, the chelator can first be functionalized using the following exemplary reaction scheme:

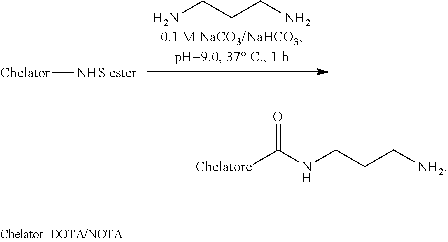

Example 1 provides a detailed synthetic protocol for preparing the radioimmunoconjugates as described herein.

The radioimmunoconjugate compounds may be prepared using the synthetic protocols disclosed herein and routine modifications thereof, which will be apparent given the disclosure herein and methods well known in the art. One of skill in the art will appreciate that other synthetic routes may be employed for preparation of the radioimmunoconjugate products and intermediates thereof. Conventional and well-known synthetic methods may be used in addition to the teachings herein. The synthesis of typical compounds and conjugates described herein may be accomplished as described in the following examples. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein.

Materials for the synthetic protocols disclosed herein are generally known compounds or can be prepared by known procedures or obvious modifications thereof. For example, the materials used in the preparation of the radioimmunoconjugates described herein are available from commercial suppliers such as Biopharma PEG (Biochempeg) Scientific Inc. (Watertown, MA, USA), Quanta Biodesign Ltd. (Plain City, Ohio, USA), Aldrich Chemical Co. (Milwaukee, Wisconsin, USA), Merck Millipore Ltd. or Millipore Sigma (Burlington, MA, USA), National Isotope Development Cente, Los Alamos National Laboratory (New Mexico, USA), Sino Biological US Inc. (Chesterbrook, PA, USA), BioLegend (San Diego, CA, USA), Sigma-Aldrich (St. Louis, Missouri, USA), or Thermo Fisher Scientific. Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5ₜₕ Edition, 2001), and Larock's Comprehensive Organic Transformations (VCH Publishers Inc., 1989).

### EXAMPLES

### Example 1: Synthesis of ²²⁵Ac-Macropa-YS5

The reaction scheme for the synthesis of ²²⁵Ac-Macropa-PEG_{4,8}-YS5 is set forth in Scheme 1, infra.

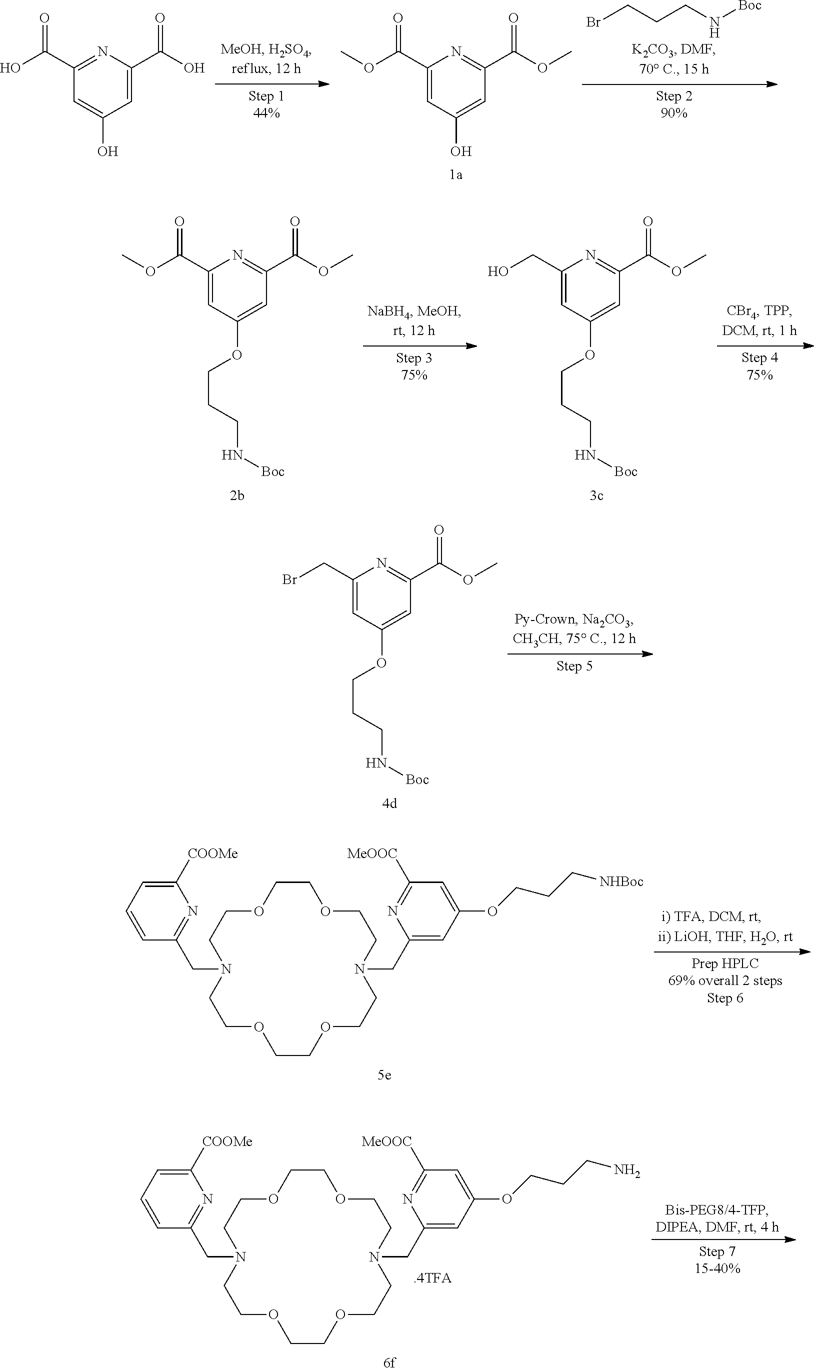

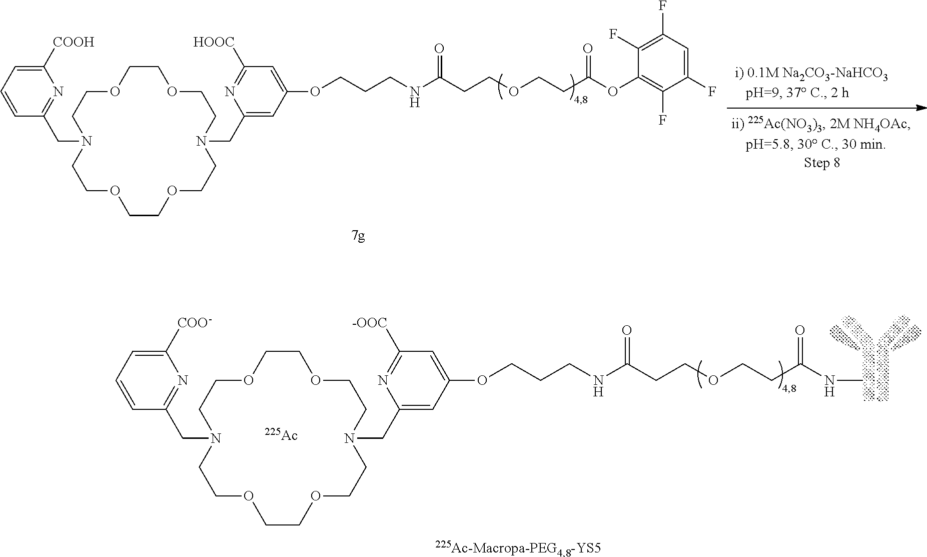

### Step 1: Synthesis of dimethyl 4-hydroxypyridine-2,6-dicarboxylate (2a)

Chelidamic acid mono hydrate (5 g, 27.32 mmol) was added 1 mL of H₂SO₄ at room temperature and the reaction mixture was refluxed for 4 h. The excess methanol was removed under reduced pressure. Then, the residue was neutralized with sat. NaHCO₃ solution under cooling. The precipitated solid was filtered, washed with water, and dried in vacuo to afford 2.4 g (44%) of compound 1a as a pale brown solid.

### Step 2: Synthesis of dimethyl 4-(3-((tert-butoxycarbonyl)amino)propoxy)pyridine-2,6-dicarboxylate (2b)

A mixture of Dimethyl 4-hydroxypyridine-2,6-dicarboxylate, la (0.5 g, 2.37 mmol) and K₂CO₃ (0.64 g, 4.74 mmol) and tert-butyl (3-bromopropyl) carbamate (0.677 g, 2.84 mmol) in DMF (6 mL) was stirred at 70 °C under a Nitrogen (N₂) condition for 12 h. The DMF was removed under reduced pressure and water was added and extracted with ethyl acetate. The organic layer was dried over anhydrous sodium sulfate (Na₂SO₄) and concentrated under reduced pressure. The crude product was purified by column chromatography over silica gel (230-400 mesh) using 90%-100% EtOAc in hexane to give 0.78 g (90%) of compound 2b as a white solid.

### Step 3: Synthesis of methyl 4-(3-((tert-butoxycarbonyl)amino)propoxy)-6-(hydroxymethyl)picolinate (3c)

NaBH₄ (62 mg, 1.63 mmol) was added in two portions to a stirred solution of compound 2b (0.5 g, 1.36 mmol) in DCM:MeOH (2:1, 20 mL) at room temperature (rt) under N₂ atmosphere and stirred for 3 h at rt. The reaction was quenched with sat. NH₄Cl and the solvents were removed under reduced pressure. The resulting residue was extracted into EtOAc, washed with brine solution, dried over anhydrous Na₂SO₄ and the organic layer was removed under reduced pressure. The crude product was purified by column chromatography over silica gel (230-400 mesh) using 5% methanol in EtOAc as an eluent to give 0.32 g (75%) of compound 3c as off-white solid.

### Step 4: Synthesis of methyl 6-(bromomethyl)-4-(3-((tert-butoxycarbonyl)amino)-propoxy)picolinate (4d)

A solution of PPh₃ (0.296 g, 1.12 mmol) was added in portion wise to a stirred solution of compound 3c (0.32 g, 0.94 mmol), CBr₄ (0.374 g, 1.13 mmol) and K₂CO₃ (0.195 g, 1.42 mmol) in CH2Cl2 (25 mL) at 0 °C (under N₂) and stirred for 1 h at rt. The reaction mixture was concentrated under reduced pressure and purified by column chromatography over silica gel (230-400 mesh) using 5% methanol in EtOAc as an eluent to give 0.28 g (75%) of compound 4d as a white solid.

### Step 5: Synthesis of methyl 4-(3-((tert-butoxycarbonyl)amino)propoxy)-6-((16-((6-(methoxycarbonyl)pyridin-2-yl)methyl)-1,4,10,13-tetraoxa-7,16-diazacyclooctadecan-7-yl)methyl)picolinate (5e)

Synthesis of Py-Crown: Methyl 6-(chloromethyl) picolinate (0.5 g, 2.7 mmol) in dry CH₃CN (60 mL) was added dropwise over 3 h to a stirred solution of 1,7,10,16-tetraoxa-4,13-diazacyclooctadecane (1 g, 3.8 mmol) and diisopropylethylamine (0.6 mL, 3.3 mmol) in dry CH₃CN (0.5 L) at 75 °C and stirred at same temperature for 48 h. The reaction mixture was concentrated under reduced pressure. The resulting crude was diluted with diethyl ether, a white precipitate was filtered and the filtrate was concentrated under reduced pressure to afford dark gold colored gummy solid. which was used in the next step of the synthesis without any further purification; A mixture of the dark brown gummy solid (1 g, 2.44 mmol), compound 4d (1.08 g, 2.68 mmol) and diisopropylethylamine (0.129 g, 6.1 mmol) in dry CH₃CN (100 mL) was stirred at 75 °C for 12 h. The solvent was removed and purified by column chromatography over neutral alumina using 5-10% methanol in dichloromethane as an eluent to give 1.1 g of compound 5e as brown viscous liquid.

### Step 6: Synthesis of 4-(3-aminopropoxy)-6-((16-((6-carboxypyridin-2-yl)methyl)-1,4,10,13-tetraoxa-7,16-diazacyclooctadecan-7-yl)methyl)picolinic acid (6f)

Compound 5e (1.1 g, 1.50 mmol) was dissolved in aq 6M HCl (15 mL) and stirred at room temperature for 3 h. After completion of the starting material (evidenced by LCMS), aq. HCl was removed under reduced pressure and the crude compound (1.4 g) was used in the next step of the synthesis without any further purification. The crude deboc product was re-dissolved in THF: 2M LiOH (15:15 mL) and stirred at rt for 12 h. The resulting crude product was purified by prep HPLC afforded 1.1 g (69%) of compound 6f as a pale brown solid.

### Step 7: Synthesis of Macropa-PEG_{4,8}-TFP ester (7g)

DIPEA (0.15 mL, 0.85 mmol) was added to a solution of compound 6f (0.15 g, 0.14 mmol) and TFP-PEG4-TFP (0.1 g, 0.17 mmol) in DMF (0.5 mL) at rt and stirred for 12 h at rt. Reaction progress was monitored by LCMS. After completion of the reaction, the rm was directly purified by prep HPLC to afford PEG4 derivative of compound 7g (61 mg, 41%) and PEG8 of compound 7g (65 mg, 11%) as gummy solid.

### Step 8: Antibody Conjugation and Radiolabeling

YS5 (1 mg) in HEPES buffer was exchanged with 0.1 M Na₂CO₃/NaHCO₃ buffer (pH=9.0) using YM30K centrifugal filter unit (Amicon ultra-0.5 mL, Regenerated cellulose, Merck Millipore Ltd.,). It was further diluted with 0.1 M Na₂CO₃/NaHCO₃ buffer (pH=9.0) to adjust the final volume of 0.2 mL. 15 eq. of PEG4 & PEG 8 of compound 7g in DMSO was added to the above solution containing 1 mg of YS5 and then incubated at 37 °C for 1 h. The reaction mixture was purified over the PD10 gel filtration column by eluting with 0.25 M NaOAc buffer, pH=6. Macropa-NCS was synthesized and conjugated with YS5 using same protocol to give Macropa-PEG₀-YS5 conjugate; Radiolabeling: 1 mCi of ²²⁵Ac(NO₃)₃ was received from Los alamos national laboratory in solid form, and it was dissolved in 0.2 M HCl solution. The radiolabeling was performed by incubating the 50 μCi (6 μL) of ²²⁵Ac(NO₃)₃, 2 M NH₄OAc (50 μL, pH=5.8), L-Ascorbic acid (20 uL, 150 mg/mL), and YS5-PEG_{0,4,8}-Macropa (200 μg) at 40 °C for 2 h. The radiolabeling progress was monitored by radio TLC by eluting with a mobile phase 10 mM EDTA (pH=5.5) on iTLC-SG with a purity of 83.39%. Then the antibody conjugation was diluted with 300 μL of 0.9% saline and centrifuged using YM30K centrifugal filtration. Then, it was washed with 0.9% saline for three times afforded ²²⁵Ac-Macropa-PEG0,4,8-YS5 (Yield: 40-60) with a radiochemical purity>95%.

It will be understood by those skilled in the art that other Chelator-Linker-Antibody compounds of the present invention can be made using a similar synthetic scheme and by following a similar synthetic protocol.

### Example 2: Study to Determine the Influence of Short PEG Linkers on Biodistribution of ²²⁵Ac-Macropa-YS5, an Immunoconjugate for Treating CD46 Expressing Cancer

In this example, the influence of PEG4 and PEG8 linkers on the biodistribution of ²²⁵Ac-Macropa-PEG₀-YS5, an antibody (Ab) targeting a cancer-specific epitope on CD46, in prostate tumors is demonstrated.

Methods: Macropa.NCS [3] and Macropa-PEG4,8-TFP esters were synthesized and subsequently coupled to YS5 IgG (Scheme 2, infra). Macropa-PEG_{0,4,8}-YS5 conjugates were prepared with different equivalents of TFP ester and purified by PD-10 gel filtration. The chelator/antibody (Ab) ratios were determined by matrix-assisted laser desorption/ionization-mass spectrometry. ²²⁵Ac (DOE) radiolabeling proceeded in 1 M NH₄OAc at 30 °C followed by YM30K centrifugal purification. Radiochemical yield (RCY) was determined by radio ITLC using 10 mM EDTA at pH 5.5. Biodistribution (0.0185 MBq, 0.5 μCi) was tested in nude mice(nu/nu) bearing prostate 22Rv1 xenografts.

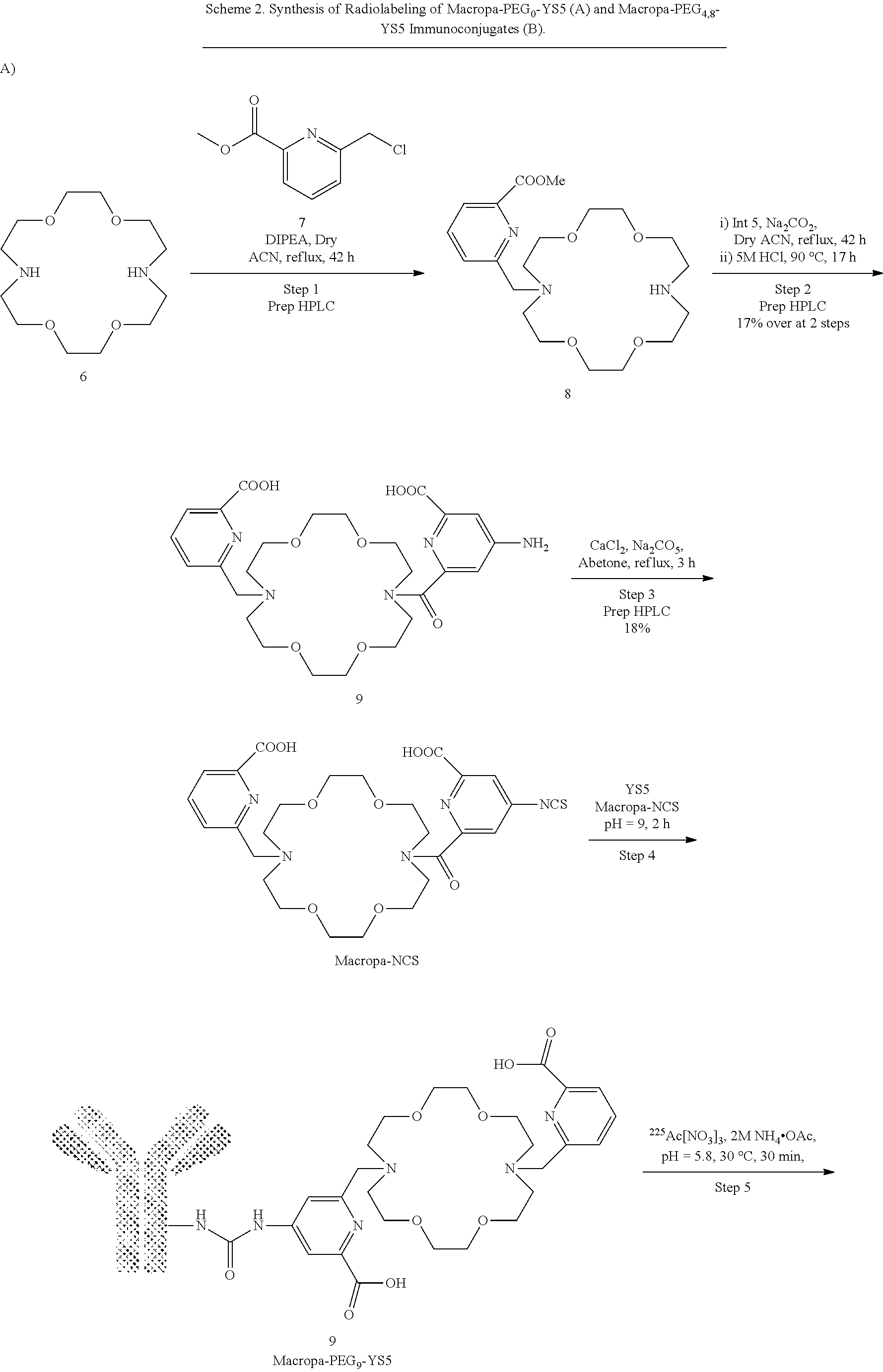

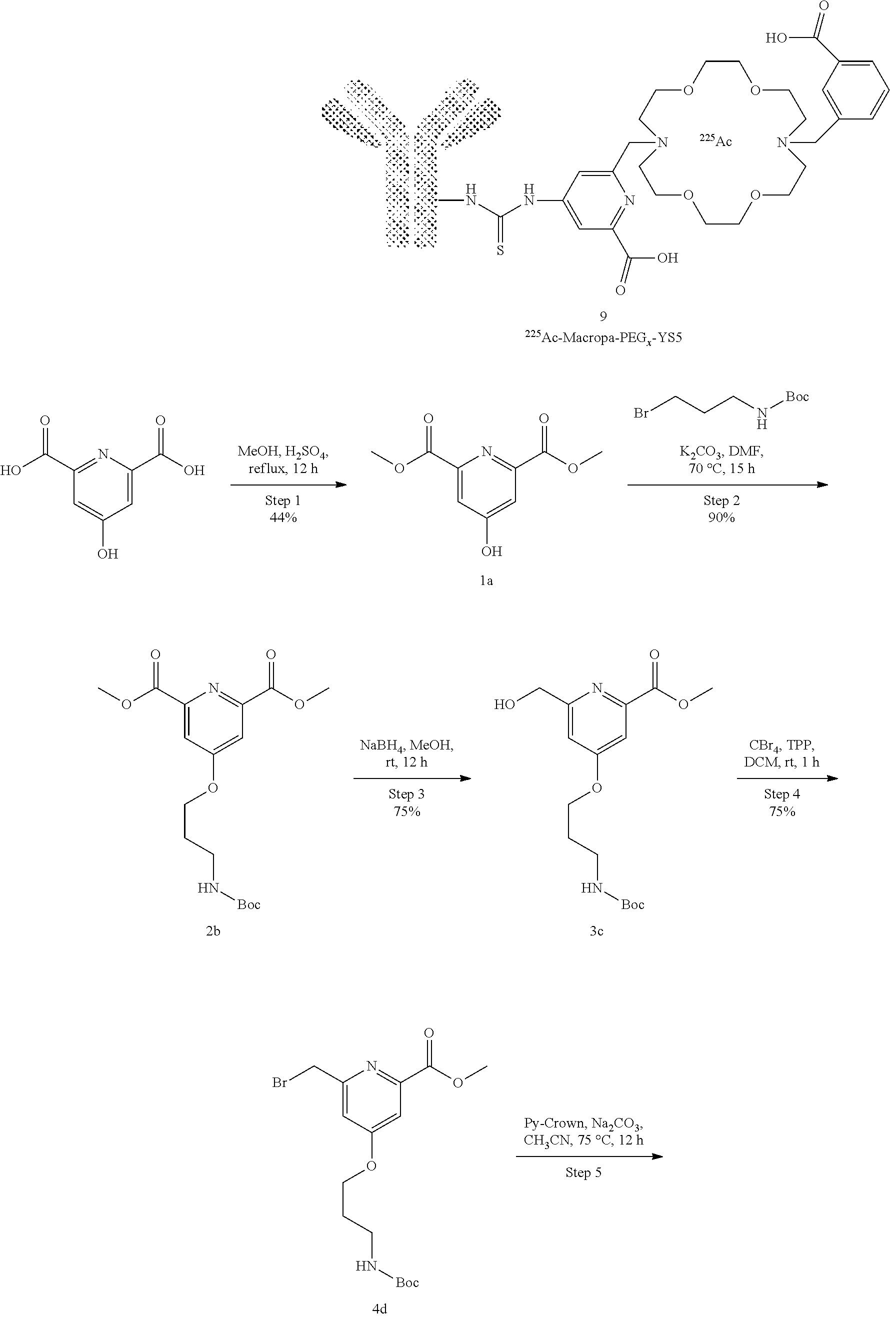

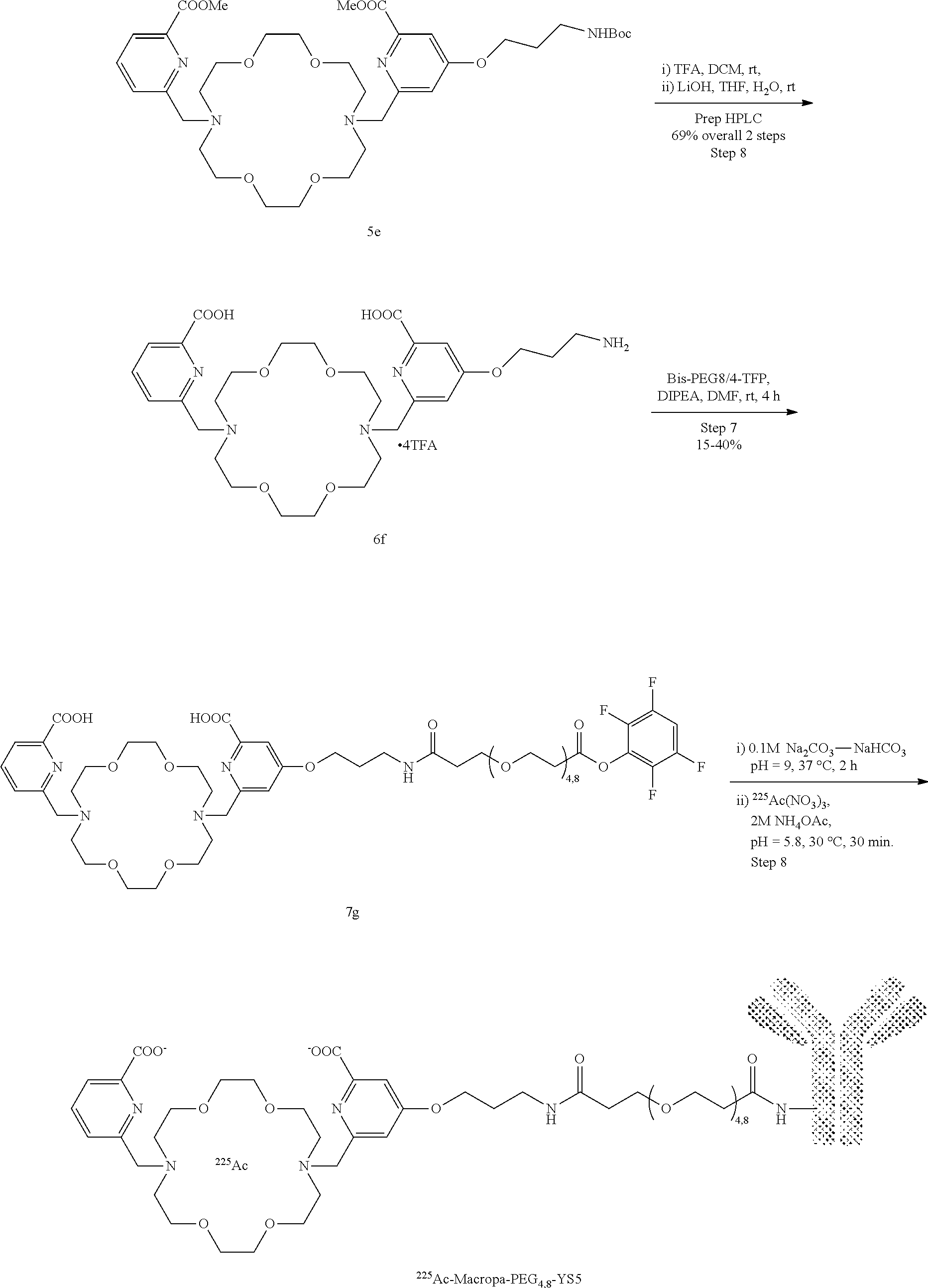

The Target Binding Fraction Assay using magnetic beads was carried out as follows: The Vials were divided into three groups named group A (Testing), group B (Blocking), and group C (Control Group). 40 mL of Hispur Ni-NTA magnetic beads (Catalog No. 88831, Thermo Fisher Scientific) were added to the vials in each group A, B and C, diluted with 380-mL phosphate-buffered saline containing 0.05% Tween-20 (PBS-T). The samples were vortexed for 15-30 seconds, and the beads were trapped using the DynaMag-2 magnet (Purchased from Thermo Fisher Scientific., Catalog number. 12321D) and the supernatant was removed. Add 5 μg of CD46 (10 μL, 25 mg/mL) per vial, PBS-T (390 uL) to groups A and B and incubated for 15 min at room temperature and the supernatant was removed. Finally, 10 ng of the ²²⁵Ac-Maropa-PEG_{0,4,8}-YS5 in 1% milk PBS (10 μL) was added to groups A, B, and C. A large excess of cold YS5 (10 μg) was added for the blocking group B before adding the 10 ng of ²²⁵Ac-Maropa-PEG_{0,4,8}-YS5. Each group was diluted with 1% milk PBS to a total volume of 400 uL/vial and incubated at room temperature for 30 min. Thereafter, the beads were isolated using a magnet, and the supernatant was removed with a pipette and collected in separate tubes. The beads were washed twice with 400 μL of PBS-T. The bead's activity, supernatant, and standard (10 ng of ²²⁵Ac-Maropa-PEG_{0,4,8}-YS5) were measured on a Hidex gamma counter. The binding fraction percentage was determined by calculating the bead's activity/standard.

Results: The Macropa chelator developed by Thiele et al. (Thiele N A et al. (2017) Angew Chem Int Ed Engl, 56(46), 14712-14717) was modified by incorporating PEG linkers and subsequently coupled to YS5 IgG. Greater than 95% radiochemical yield was achieved when labeling Macropa-PEG_{0,4,8}-YS5 with ²²⁵Ac, regardless of chelator and regardless of chelator ratios ranging from 1 to 7.76 per YS5 antibody (n=2), and the isolated yield was greater than 60% after purification (radiochemical purities>98% (see, Table 10, infra).

TABLE 9
Radiolabeling of Macropa-PEG_{0, 4, 8}-YS5 with
²²⁵Ac(NO₃)₃, (1:4 ratio of ²²⁵AcᵥₛYS5)

| Conjugate | RCY | Isolated | SA | No. of |
|---|---|---|---|---|
| Name | (%) | Yield (%) | (μCi/ug) | trials |
| PEG₀(4.5) | 96.67 ± 0.27 | 80 | 0.2 | 2 |
| PEG₄(2.2) | 96.79 | 68.33 | 0.17 | 1 |
| PEG₈(7.76) | 100 | 67.5 | 0.17 | 1 |
| PEG8(1) | 96.62 | 55 | 0.14 | 1 |
| DOTA (8.5) | 81.4 ± 3.3 | 40.6 ± 1.6 | 0.1 | 3 |

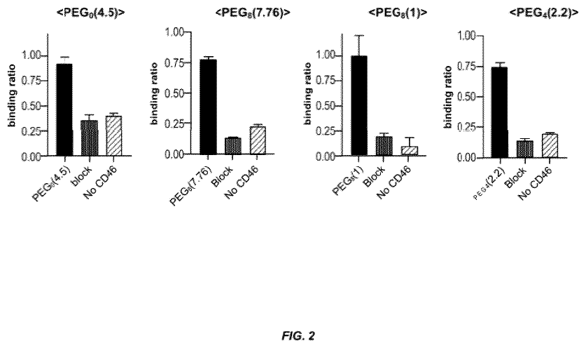

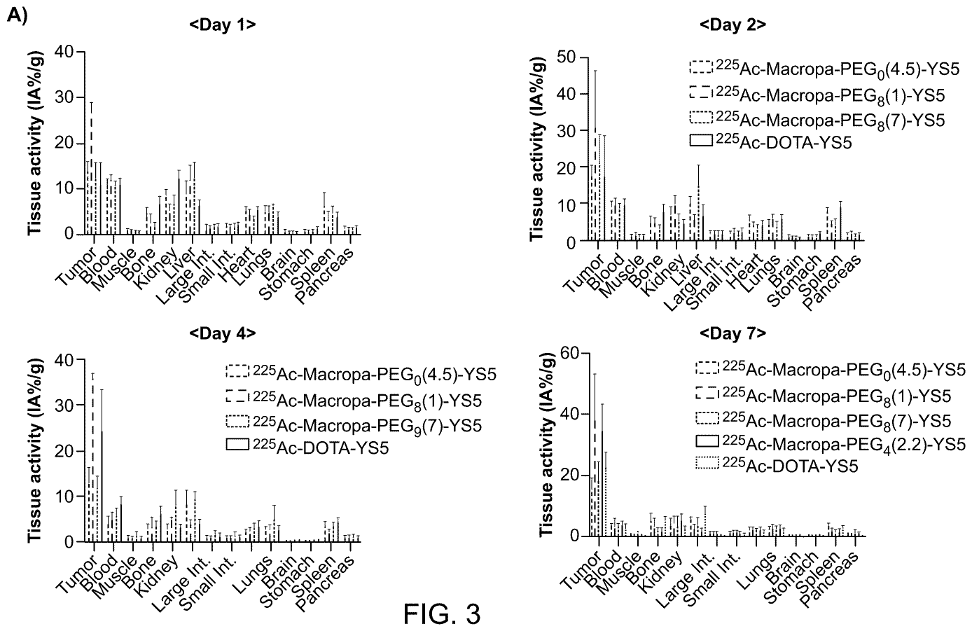

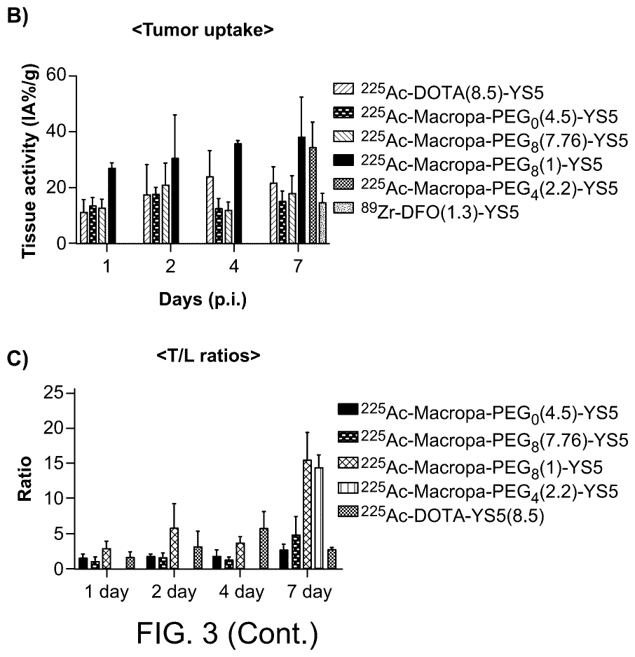

Such high conversions were not seen with DOTA-YS5 conjugate even at a higher ratio of 8.5 chelators per antibody (RCY~83%, ~40% isolated yield, and RCP~95%). The target binding fraction for ²²⁵Ac-Macropa-PEG_{0,4},8-YS5 conjugates against CD46 showed >75% binding for all the radioimmunoconjugates (see, FIG. 2). The effect of PEG linkers was evaluated via biodistribution in 22Rv1 prostate cancer xenografts (see, FIGS. 3A-3C). Biodistribution at 7 d post-injection showed high tumor uptake of 34.2 and 38.2% ID/g for PEG4 and PEG₈, respectively, compared to 15.5 & 21.6% ID/g for the non-PEGylated conjugate and DOTA. Reduced tumor uptake of 18.5% ID/g for ²²⁵Ac-Macropa-PEG8-YS5 (7.76) was observed, which may be due to the presence of a higher chelator ratio (~7.76) per antibody. ⁸⁹Zr-DFO-YS5 with ~1.3 chelators per antibody (Wang S. et al., (2021) Clin Cancer Res, 27(5), 1305-1315) showed comparable tumor uptake (14.5% ID/g) to the non-PEGylated ²²⁵Ac-Macropa-PEG0-YS conjugate, however, a significant (P<0.05) increase in tumor uptake was shown for the PEGylated conjugates ²²⁵Ac-Macropa-PEG_{4,8}-YS5 with ~2.2 & ~1 chelators per antibody, respectively.

Conclusions: Higher radiolabeling conversions were achieved for ²²⁵Ac-Macropa-PEG_{0,4,8}-YS5 conjugates in comparison with DOTA-YS5 conjugate regardless of chelator ratios per antibody. The biodistribution results demonstrated that the insertion of PEG linkers enhances tumor uptake (up to 2-fold) in contrast to non-PEGylated conjugates. Taken together, these data demonstrate that optimized PEGylated Macropa based chelators represent an improved radiolabeling platform for radioimmunotherapy using ²²⁵Ac.

### Example 3: Preparation and Analysis of Macropa-PEG_{0,4,8}-YS5 Conjugates

### Synthesis of Macropa-PEG0,4,8-YS5 Conjugates

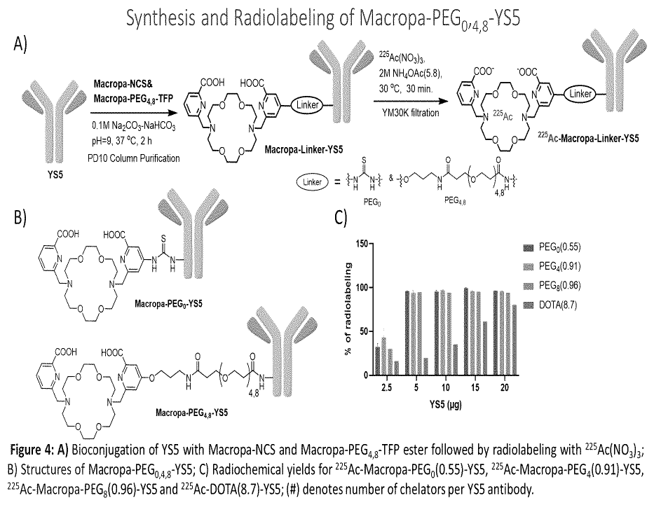

Macropa NCS was synthesized according to the reported literature. To insert PEG4 and PEG₈ linkers, a new analog of bifunctional chelator Macropa (Intermediate 6f) and Macropa-PEG₄-TFP ester was synthesized in our lab and reported in our previous communication. Macropa-PEG₈-TFP ester was synthesized by reacting the intermediate 6f in the presence of DIPEA in DMF followed by prep HPLC purification (Yield~34%) (Reaction scheme, FIG. 4). With these derivatives are in hand, a typical standard methodology is used to conjugate the Lysine residue on the antibody YS5. The initial attempts resulted in different chelators per antibody YS5 as the conjugation was non-specific. Nearly 1:1 ratios of chelator per antibody YS5 were obtained by varying the number of equivalents of Macropa-NCS and Macropa-PEG_{4,8}-TFP esters. MALDI-TOF MS estimated the number of chelators.

### Radiolabeling and In Vitro Studies

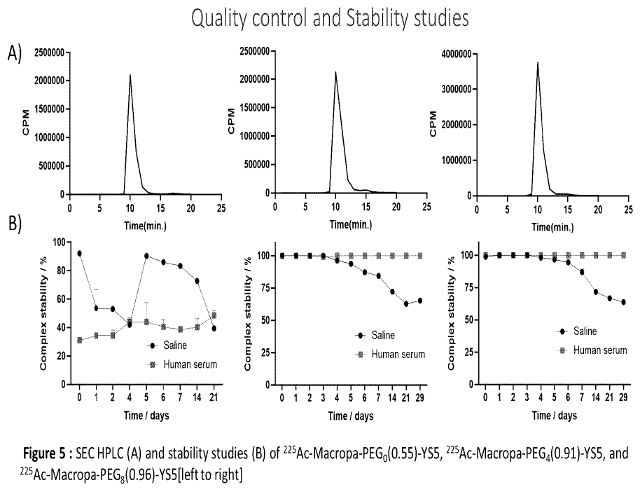

At first, the radiolabeling efficiency of synthesized conjugates with nearly 1:1 chelator per antibody ratios of Macropa-PEG_{0,4,8}-YS5 conjugates was evaluated with different molar ratios and compared to its DOTA-YS5 conjugate (8.7 chelators per YS5). Radiolabeling was performed by incubating the various amounts of Macropa-PEG_{0,4,8}-YS5 (2.5, 5, 10, 15, 20 μg) with Ac-225 (5 μCi) in 2 m NH₄OAc, pH=5.8 at 30 °C for 30 min. However, DOTA-YS5 was set for 2 h at 40 °C. As expected, >94% radiochemical yields (RCY) were noticed for Macropa-PEG_{0,4,8}-YS5 conjugates starting from 1:1 to 1:4 metal-to-antibody ratios. In contrast, DOTA-YS5 shows RCY~79% even at a high temperature (40 °C) and time (2 h) (FIG. 4). The radiochemical yields were slightly dropped for Macropa-PEG₀(0.55)-YS5 in comparison with Macropa-PEG_{4,8}(0.91, 0.96)-YS5. The radiochemical yields almost matched with Macropa-PEG_{4,8}(0.91, 0.96)-YS5 when the number of chelators was corrected to 1:1 ratio for Macropa-PEG₀(0.55)-YS5. Radio iTLC-SG is used for monitoring the labeling kinetics. The 1:4 metal-to-antibody ratio was chosen for further radiolabeling to perform both in vitro and in vivo studies. Table 10, supra, lists the radiochemical yields before or after purification, isolated yields, and specific activities for all the radioimmunoconjugates, along with abbreviated names used. The nearly 1:1 ratios of chelator to antibody conjugates PEG₀(0.55), PEG₄(0.91), and PEG₈(0.96) were subjected to size-exclusive HPLC and show no signs of aggregation. Next, the stability of the Ac-225 labeled PEG₀(0.55), PEG₄(0.91), and PEG₈(0.96) was exposed to 0.9% sterile saline and human serum at 37 °C for 29 days, and the decomplexation was monitored by radio iTLC(n=2). As shown in FIG. 5, PEG₄(0.91) and PEG₈(0.96) showed 100% complex stability in human serum, whereas PEG₀(0.55) was decomplexed even at 0 h and maintained until 21 days. In saline, PEG₄(0.91) and PEG₈(0.96) were stable until three days, then the decomplexation started slowly, starting from 3 days to 29 days (FIG. 5). In contrast, a drastic fallout was observed for PEG₀(0.55) from day 1 to day 4. Surprisingly, day 5 showed >95% stability, and then again, decomplexation was noticed over time and showed % stability at day 21. It is hypothesized that the YS5-PEG₀-Macropa degraded to Macropa.NH2 at day 5 (showed 100%) and further decomplexation was observed from the 225Ac-Macropa-NH2. To prove the hypothesis, the day 5 reaction mixture was subjected to SEC, compared with the freshly prepared 225Ac-Macropa.NH2 retention time(rt) and were well-matched with each other. The DOTA(7.7) has similar chemistry as PEG₀(0.55), however, DOTA(7.7) showed similar stability as PEG₄(0.91) and PEG₈(0.96). Overall, these results demonstrated that PEG₀(0.55) is unstable in both saline and human serum in comparison with PEG₄(0.91) and PEG₈(0.96) conjugates.

### Biodistribution

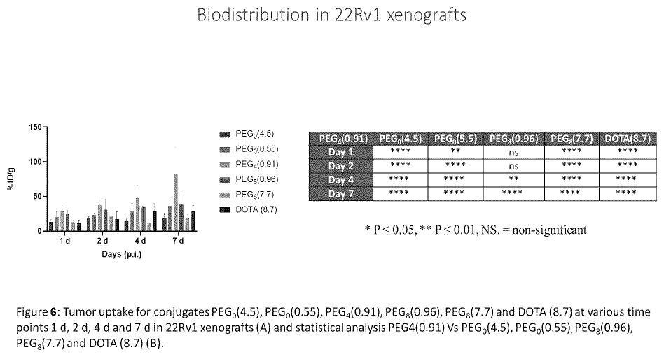

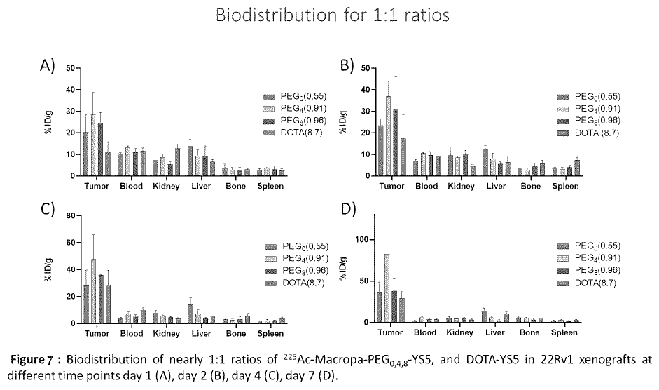

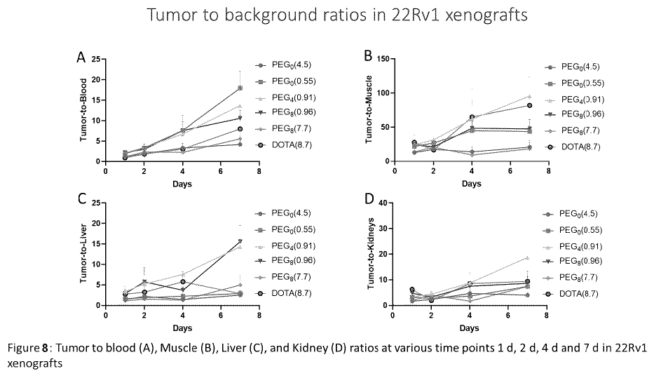

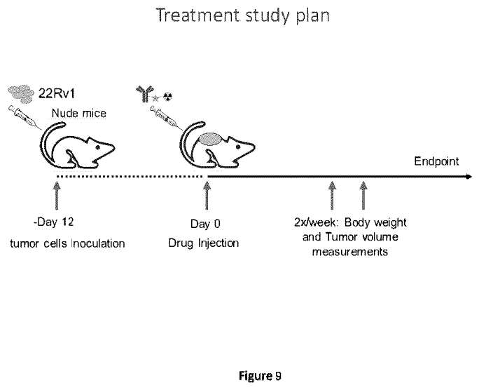

The influence of the short PEG linkers was evaluated through ex-vivo biodistribution for all the radioimmunoconjugates 225Ac-Macropa-PEG_{0,4,8}-YS5 in 22Rv1 tumor-bearing mice (0.5 μCi in saline), and the uptakes were compared with each other as well as 225Ac-DOTA(8.7)-YS5, a radioimmunoconjugate that has thoroughly been studied for its therapeutic efficacy in 22Rv1 xenografts in our lab (see, FIGS. 6-7). The injected dose (% ID) per organ was quantified by dividing the decay-corrected percent of the injected dose. The tumor uptake for all the conjugates gradually increased over time, regardless of the PEG linkers and chelators per antibody YS5, and slowly cleared from the non-targeted organs. The higher tumor uptake for PEG4(0.91) showed at all the time points (28.54±10.40, 37.09±6.99, 47.85±18.18, and 82.82±38.27%) in comparison with PEGylated conjugate PEG8(0.96) (24.64±4.92, 30.79±15.18, 35.98±0.45, and 38.15±14.41%), non-PEGylated conjugates PEG₀(0.55) (20.31±8.02, 23.49±2.89, 28.24±11.44, 36.39±12.4%) and DOTA (8.7). Although the tumor uptakes for PEG₀(0.55) were comparable with PEG8(0.96), simultaneously, liver accumulation was also high (12.97±4.5% at day 7) at all the time points in comparison with PEG4(0.91) (6.04±1.72% at day 7) and PEG8(0.96) (2.56±1.19% at day 7) conjugates. In contrast, the PEG₀(4.5) (13.4±3.40, 18.8±2.42, 14.14±4.87, and 18.53±7.17%), PEG₈(7.7) (12.66±3.11, 21.06±7.52, 11.82±2.72, and 18.51±5.55%) showed low tumor uptakes, and high liver uptakes PEG₀(4.5) (7.3±1.62%) and PEG₈(7.7) (4.23±1.44%). This indicates the immunoreactivity of antibody YS5 was impacted due to heavy modification of antibody YS5 (more number of chelators~4.5 and ~7.7) even after PEGylation, and results demonstrate that higher tumor uptakes can be obtained by inserting the short PEG linkers with less number of chelators. The blood uptakes were significantly lowered for all the conjugates PEG₀(0.55) (2.01±0.43%), PEG₄(0.91)(5.87±0.83%), PEG₈(0.96)(3.86±1.33%), PEG₀(4.5)(4.32±1.20%), PEG₄(2.2), PEG₈(7.7) (3.4±0.60%) and DOTA(8.7)(3.93±0.94%) at 7-day post-injection. It is thought that the low blood uptake for PEG₀(0.55) is due to the instability of the conjugate in vivo, as observed in in vitro studies. Higher tumor-to-blood (13.70±4.93%), tumor-to-liver (14.29±8.02%), tumor-to-muscle (95.67±46.38%), and tumor-to-kidneys (18.70±11.29%) were observed for PEG4(0.91) in comparison with other conjugates at 7 days post-injection except for PEG₀(0.55) tumor-to-blood ratios (17.93±4.17%) (see, FIG. 8). All taken together, the data demonstrate that PEG4(0.91) is a promising radioimmunoconjugate with higher tumoral uptakes compared with other radioimmunoconjugates while reducing the activity concentration in the blood and other healthy organs (see, FIGS. 9-12).

### 225Ac-Macropa-PEG₄-YS5 is Effective in Reducing Tumor Volume and Prolonging Survival in 22Rv1 Prostate Cancer Models.

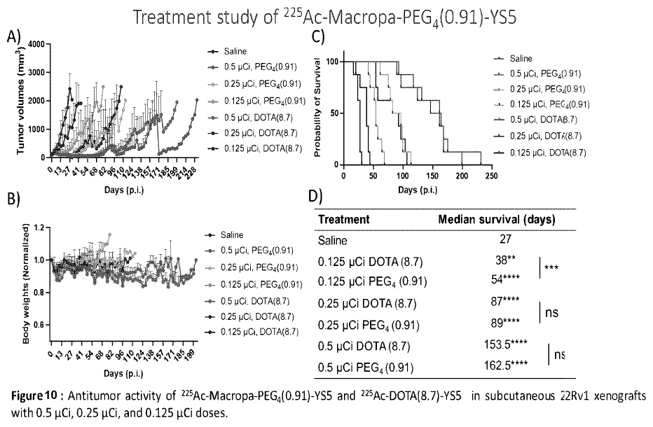

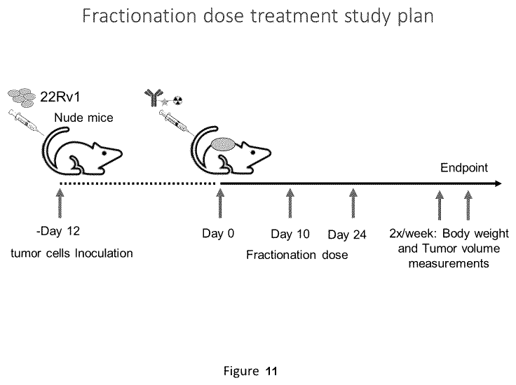

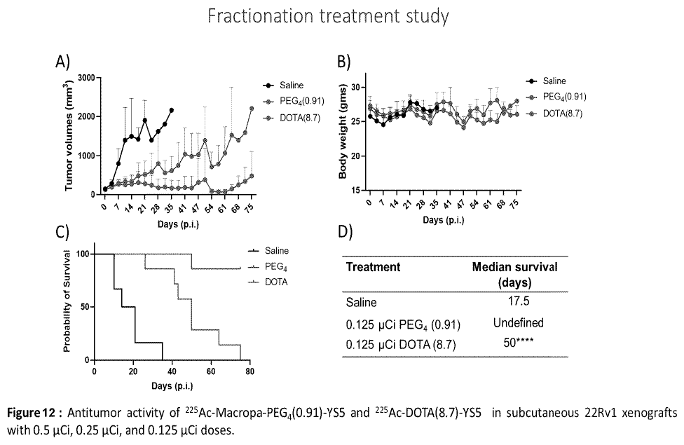

CD46-expressing cell line 22Rv1 xenograft models were used to study therapeutic efficacy of 225Ac-Macropa-PEG4-YS5 in comparison to 225Ac-DOTA-YS5. Cohorts of n=8 22Rv1 xenograft bearing mice were randomized to injection with saline, 0.25 μCi or 0.5 μCi activity levels of 225Ac-Macropa-PEG4-YS5 and 225Ac-DOTA-YS5 (FIG. 9). As shown in FIG. 10, treatment with 0.25 μCi and 0.5 μCi of [225Ac]DOTA-YS5 significantly inhibited tumor growth activity-dependent, while mice in the control saline cohort showed rapid tumor growth for both the conjugates. However, the 0.125 μCi dose of 225Ac-Macropa-PEG4-YS5 showed greater tumor growth inhibition in comparison with 225Ac-DOTA-YS5 and control saline group, and the survival plot indicates that the median survival of the mice treated with 0.125 uCi of 225Ac-Macropa-PEG4-YS5 was 54 days, 38 days for 225Ac-DOTA-YS5 (p<0.001), and control saline was 27 days (p<0.0001) respectively. A fractionated therapy regimen was also tested to further evaluate the treatment efficacy of 225Ac-Macropa-PEG4-YS5 over 225Ac-DOTA-YS5 (FIG. 11). Fractionated dose (three doses) of 0.125 μCi was administered to the mice with 22Rv1 xenografts on day 0, day 10, and day 24. As shown in FIG. 12, the three fractionated administrations of 0.125 μCi 225Ac-Macropa-PEG4-YS5 delayed tumor growth significantly compared to the 225Ac-DOTA-YS5 and saline control group. These data demonstrate that the 225Ac-Macropa-PEG4-YS5 conjugate showed improved therapeutic efficacy over 225Ac-DOTA-YS5 in fractionated and single dose therapy regimens. These data also demonstrate that optimized PEGylated Macropa-based Immunoconjugates of the present invention exhibited more prolonged antitumor activity than the DOTA conjugate, demonstrating the high potential for clinical translation of the 225Ac-Macropa-PEG4-YS5 conjugate.

What is claimed is:

1. A radioimmunoconjugate, the radioimmunoconjugate comprising:
an immunoconjugate according to Formula I: 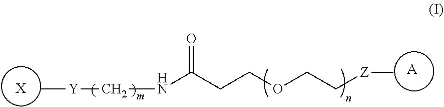
wherein:
X is a chelator moiety;
Y is selected from the group consisting of —O— and —NR—;
Z is a moiety selected from the group consisting of: 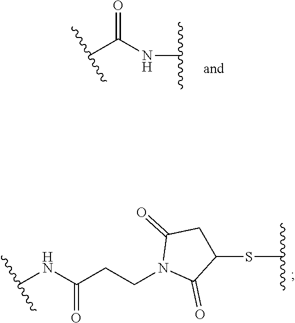 [SMILES: *NC(*)=O ; *NC(=O)CCN1C(=O)CC(S*)C1=O]
A is an antibody;
subscript m is 3 or 5;
subscript n is 4, 6, 8, 10, 12, 14, or 16; and
R is H or OH; and
a radionuclide, wherein the chelator moiety of the immunoconjugate chelates the radionuclide.

2. The radioimmunoconjugate of claim 1, wherein the immunoconjugate has a structure according to Formula Ia:

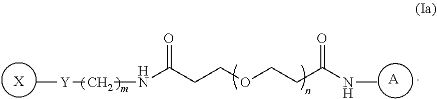

3. The radioimmunoconjugate of claim 1, wherein the immunoconjugate has a structure according to Formula Ib:

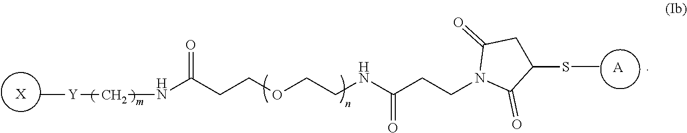

4. The radioimmunoconjugate of claim 1, wherein X is selected from the group consisting of:

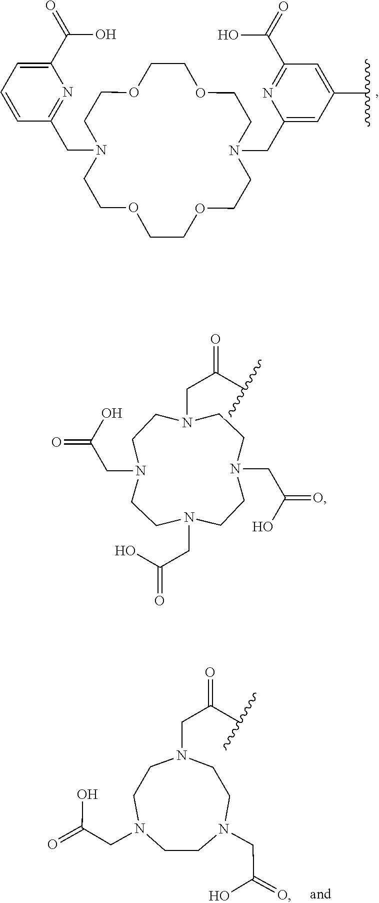 [SMILES: *c1cc(CN2CCOCCOCCN(Cc3cccc(C(=O)O)n3)CCOCCOCC2)nc(C(=O)O)c1 ; *C(=O)CN1CCN(CC(=O)O)CCN(CC(=O)O)CCN(CC(=O)O)CC1 ; *C(=O)CN1CCN(CC(=O)O)CCN(CC(=O)O)CC1]

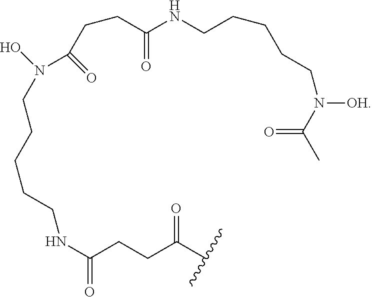 [SMILES: *C(=O)CCC(=O)NCCCCCN(O)C(=O)CCC(=O)NCCCCCN(O)C(C)=O]

5. The radioimmunoconjugate of claim 1, wherein subscript n is 4, 6, 8, or 12.

6. The radioimmunoconjugate of claim 1, wherein the radionuclide is selected from the group consisting of ²²⁵Ac, ²¹³Bi, ²²⁴Ra, ²¹²Pb, ²²⁷Th, ²²³Ra, ²¹¹At, and ¹⁴⁹Tb.

7. The radioimmunoconjugate of claim 1, wherein the radionuclide is selected from the group consisting of ¹⁷⁷Lu and ⁸⁹Zr.

8. The radioimmunoconjugate of claim 1, having a structure selected from the group consisting of:
a structure according to Formula IIa: 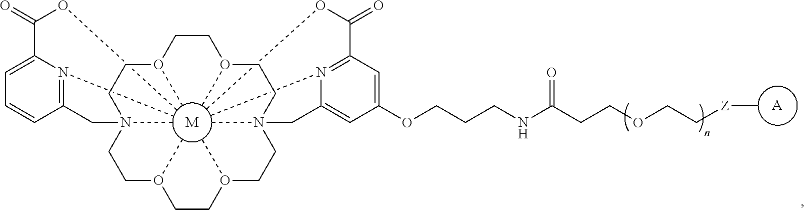
wherein M is the radionuclide;
a structure according to Formula IIb: 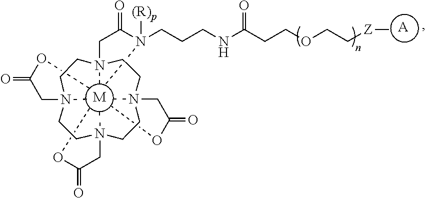
wherein M is the radionuclide, and subscript p is 0 or 1;
a structure according to Formula IIc: 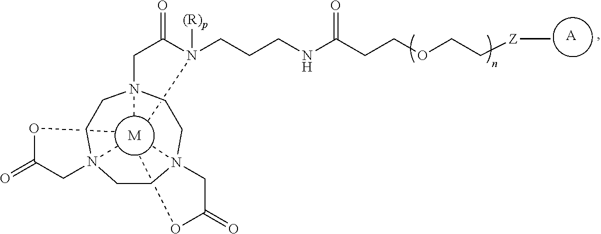
wherein M is the radionuclide, and subscript p is 0 or 1; and
a structure according to Formula IId: 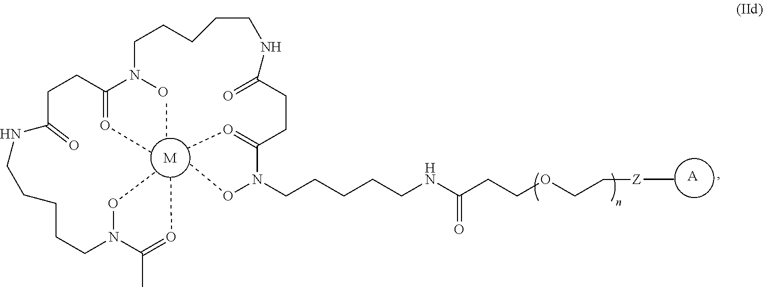
wherein M is the radionuclide.

9. A pharmaceutical composition comprising the radioimmunoconjugate of claim 1 and a pharmaceutically acceptable excipient.

10. A method of treating cancer in a subject, the method comprising administering to the subject the radioimmunoconjugate of claim 1.

11. A method of treating prostate cancer in a subject, the method comprising administering to the subject the radioimmunoconjugate of claim 1.

12. A radioimmunoconjugate precursor having the structure: 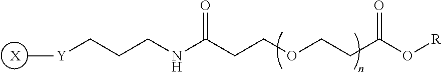
wherein:
X is a chelator moiety;
Y is O or N;
subscript n is 4, 6, 8, or 12; and
R is N-hydroxysuccinamide (NHS) or tetrafluorophenyl (TFP).

13. The radioimmunoconjugate precursor of claim 12, wherein X is selected from the group consisting of:

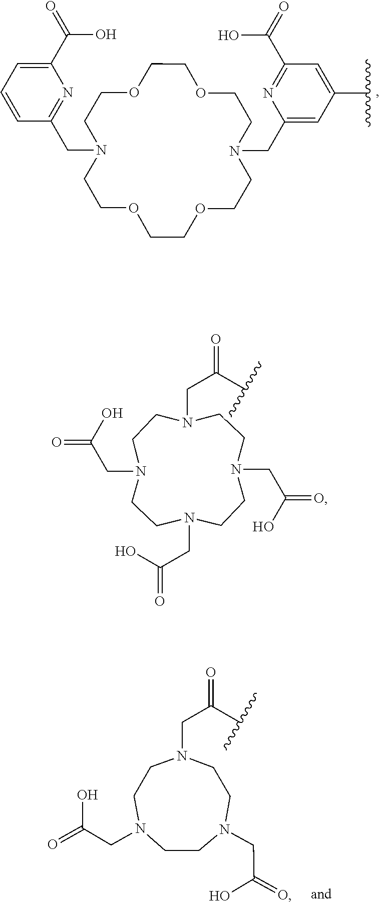 [SMILES: *c1cc(CN2CCOCCOCCN(Cc3cccc(C(=O)O)n3)CCOCCOCC2)nc(C(=O)O)c1 ; *C(=O)CN1CCN(CC(=O)O)CCN(CC(=O)O)CCN(CC(=O)O)CC1 ; *C(=O)CN1CCN(CC(=O)O)CCN(CC(=O)O)CC1]

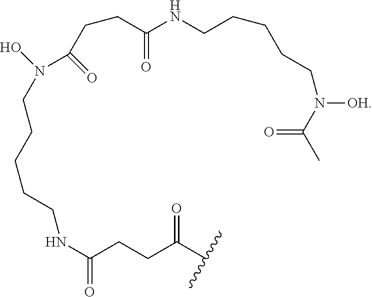 [SMILES: *C(=O)CCC(=O)NCCCCCN(O)C(=O)CCC(=O)NCCCCCN(O)C(C)=O]

14. The radioimmunoconjugate precursor of claim 12, having the structure: 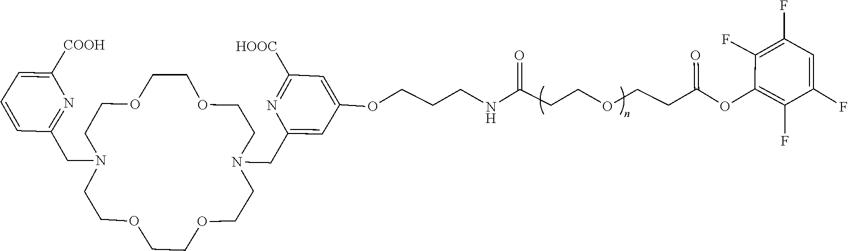
wherein subscript n is 4 or 8.

15. A radioimmunoconjugate precursor having the structure: 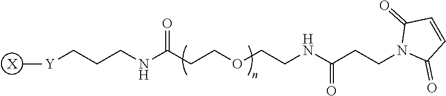
wherein:
X is a chelator moiety;
Y is O or N; and
subscript n is 4, 6, 8, or 12.

16. The radioimmunoconjugate precursor of claim 15, wherein X is selected from the group consisting of:

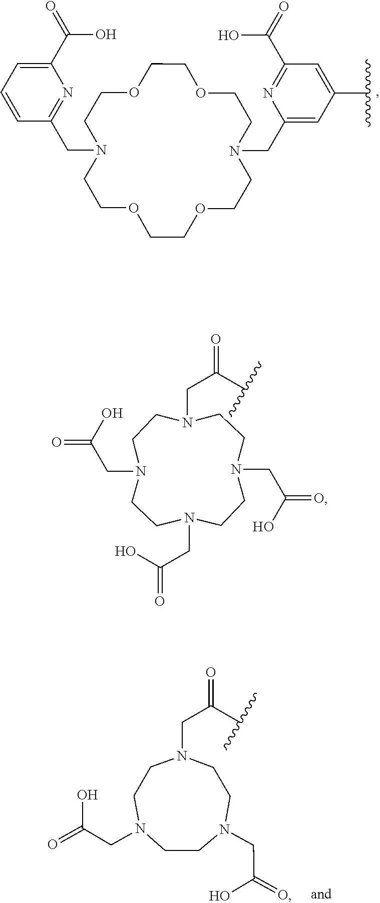 [SMILES: *c1cc(CN2CCOCCOCCN(Cc3cccc(C(=O)O)n3)CCOCCOCC2)nc(C(=O)O)c1 ; *C(=O)CN1CCN(CC(=O)O)CCN(CC(=O)O)CCN(CC(=O)O)CC1 ; *C(=O)CN1CCN(CC(=O)O)CCN(CC(=O)O)CC1]

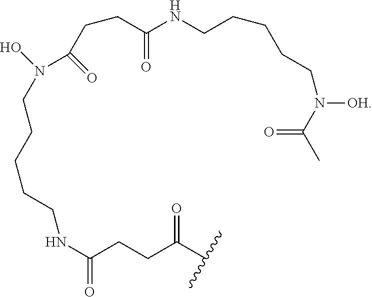 [SMILES: *C(=O)CCC(=O)NCCCCCN(O)C(=O)CCC(=O)NCCCCCN(O)C(C)=O]

17. A linker having the structure: 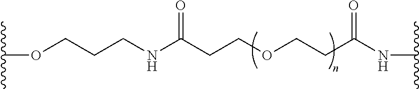
wherein subscript n is 4 or 8.
